(19) United States
(12) Patent Application Publication
(10) Pub. No.: US 2024/0077500 A1
(43) Pub. Date: Mar. 7, 2024

(54) METHOD FOR THE DETECTION AND TREATMENT OF PROTEINOPATHIES

(71) Applicant: Women & Infants Hospital of Rhode Island, Providence, RI (US)

(72) Inventor: Surendra Sharma, Warwick, RI (US)

(73) Assignee: Women & Infants Hospital of Rhode Island, Providence, RI (US)

(21) Appl. No.: 18/260,661

(22) PCT Filed: Jan. 13, 2022
(86) PCT No.: PCT/US2022/012296

§ 371 Date: Jul. 7, 2023

Related U.S. Application Data
(60) Provisional application No. 63/137,371, filed on Jan. 14, 2021.

(51) Int. Cl. G01N 33/68; G01N 33/50

(52) U.S. Cl. CPC G01N 33/6896; G01N 33/5091; C12N 2503/00; G01N 2333/4709; G01N 2800/28

(57) ABSTRACT

The present disclosure relates in part to a blood-based method for the detection of a proteinopathy in a subject including, but not limited to, Alzheimer's disease (AD), mild cognitive impairment (MCI), and preeclampsia (PE), utilizing autophagy-deficient trophoblast (ADT) cells to sequester protein aggregates from the serum of said subject, and permit detection thereof. The present disclosure further relates to methods of treating, preventing, and/or ameliorating a proteinopathy in a subject by the administration of trehalose, a salt, solvate, stereoisomer, derivative, prodrug and or any mixture thereof.

### CROSS-REFERENCE TO RELATED APPLICATIONS

The present application claims priority under 35 U.S.C. § 119(e) to U.S. Provisional Application No. 63/137,731, filed Jan. 14, 2021, which application is hereby incorporated herein by reference in its entirety.

### STATEMENT REGARDING FEDERALLY SPONSORED RESEARCH OR DEVELOPMENT

This invention was made with government support under grant number GM121298 and GM114750 awarded by the National Institutes of Health. The government has certain rights in the invention.

### SEQUENCE LISTING

The ASCII text file named “371288-3003W01 Sequence Listing,” created on January 6, 2022, comprising 1.4 Kbytes, is hereby incorporated by reference in its entirety.

### BACKGROUND OF THE INVENTION

Toxic extracellular and intracellular deposition of misfolded protein aggregates in the brain is a hallmark feature of proteinopathy in many neurodegenerative diseases such as Alzheimer's disease (AD). Additionally, it has recently been demonstrated that preeclampsia (PE), a severe pregnancy complication, mechanistically exists on the spectrum of proteinopathies. PE occurs in 3-8% of all pregnancies and is diagnosed by presentation with de novo onset of hypertension and proteinuria at or after 20 weeks of gestation. It can manifest as early (<34 weeks gestation) or late (>34 weeks gestation) onset, and present severe complications. PE represents a unique case of proteinopathy in a younger population and can also lead to chronic conditions later in life, including mild cognitive impairment (MCI) and dementia in mothers and their offspring.

Both AD and PE are devastating disorders for which there are currently no readily available, non-invasive, and/or cost-effective methods to detect the protein aggregates implicated therein, which arise in the prodromal phase of AD or at early pregnancy stage in the clinic. Thus, there is a need in the art for a cost-effective, easy to perform, blood test to detect protein aggregates indicative of proteinopathies, particularly AD, MCI, and PE. Furthermore, there is a need in the art for methods of treating, preventing, and/or ameliorating proteinopathies. The present disclosure addresses these needs.

### BRIEF SUMMARY OF THE INVENTION

In one aspect, the present invention provides method of detecting a proteinopathy in a subject, the method comprising:
incubating autophagy-deficient trophoblast (ADT) cells in a medium comprising a serum sample collected from the subject to provide ADT cells comprising at least one protein aggregate;
staining the cells comprising at least one protein aggregate with a protein aggregate dye to provide stained cells; and
detecting the at least one protein aggregate in the stained cells.

In certain embodiments, the proteinopathy is at least one selected from the group consisting of Alzheimer's disease (AD), mild cognitive impairment (MCI), preeclampsia (PE), Lewy body dementia (LBD), gestational diabetes, and Huntington's disease (HD).

In certain embodiments, the ADT cells comprise immortalized HchEpC1b human extravillous trophoblast cells. In certain embodiments, the ADT cells comprise human trophoblast TCL1 or HTR8 cells.

In certain embodiments, the at least one protein aggregate comprises at least one protein selected from the group consisting of transthyretin (TTR), amyloid R (Aβ), α-synuclein (α-syn), Tau231 (T231), and cis P-Tau.

In certain embodiments, the staining step comprises the following:
(a) treating the cells comprising the at least one protein aggregate with a phosphate buffered saline (PBS) solution comprising formaldehyde to provide fixed cells;
(b) incubating the fixed cells with glycine to provide quenched cells;
(c) incubating the quenched cells with a permeabilizing solution to provide permeabilized cells;
(d) washing the permeabilized cells to provide washed cells; and
(e) incubating the washed cells with the protein aggregate dye to provide stained cells.

In certain embodiments, the protein aggregate dye is ProteoStat dye.

In certain embodiments, detection of the at least one protein aggregate in the stained cells comprises measuring at least one optical property of the stained cells. In certain embodiments, the at least one optical property is fluorescence.

In another aspect, the present invention provides a method of treating, preventing, and/or ameliorating a proteinopathy in a subject in need thereof, the method comprising administering to the subject a pharmaceutical composition comprising a therapeutically effective amount of trehalose or salt, solvate, stereoisomer, derivative, prodrug and any mixtures thereof.

In certain embodiments, the trehalose stereoisomer is lactotrehalose. In certain embodiments, the proteinopathy is selected from the group consisting of Alzheimer's disease (AD) and mild cognitive impairment (MCI).

In certain embodiments, the trehalose administration promotes degradation of at least one protein aggregate. In certain embodiments, the at least one degraded protein aggregate comprises at least one protein selected from the group consisting of transthyretin (TTR), amyloid β(Aβ), α-synuclein (α-syn), Tau231 (T231), and cis P-Tau.

In certain embodiments, the trehalose is administered to the subject weekly. In certain embodiments, the trehalose is administered to the subject for a period of one month. In certain embodiments, the therapeutically effective amount is about 2 g/kg based on subject weight.

In certain embodiments, the pharmaceutical composition comprises trehalose and at least one pharmaceutically acceptable carrier.

In certain embodiments, the subject is co-administered at least one additional agent useful for treating, preventing, and/or ameliorating a proteinopathy in a subject.

In certain embodiments, the at least one additional reagent useful for treating, preventing, and/or ameliorating a proteinopathy is selected from the group consisting of transthyretin (TTR) and human chorionic gonadotropin (hCG).

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1A provides a schematic diagram of the protocol for the protein aggregation assay. Autophagy-deficient trophoblast cells (ADT) were incubated for various periods of time with aggregate-containing sera, cells are fixed, then cells are stained with ProteoStat dye alone or in combination with specific protein immunostaining antibodies. The ProteoStat fluorescent signal was imaged with a laser confocal microscope and quantified.

FIG. 1B provides representative images for the internalization and accumulation of in vitro generated transthyretin (TTR) aggregates in ADT. TTR aggregates were generated by incubation of recombinant human TTR in acetate buffer. ADT were treated with native TTR or aggregated TTR, fixed at 0 h or overnight after 20 min incubation on ice, then stained with ProteoStat dye. The nuclei were stained with DAPI. Reference bar: 20 μm; indicated by bar in lower right quadrant of Aggregated TTR images.

FIG. 2 provides a bar graph demonstrating the validation of in vitro generated TTR aggregates. Substantially higher levels of protein aggregates were detected in the solution containing in vitro generated TTR aggregates and commercial lysozyme aggregates (Enzo) compared with BSA, native lysozyme, and native TTR (p<0.001). Protein aggregates were detected from indicated samples using Proteostat protein aggregation assay kit.

FIG. 3 provides confocal microscopy images showing no accumulation of protein aggregates in ADT treated sera from non-pregnancy and normal pregnancy. ADT were incubated with 10% of sera from non-pregnancy, normal pregnancy (NPS), or preeclampsia (PES) as positive controls, fixed at 24 h, and stained with ProteoStat dye. Images are representatives of at least 3 independent experiments. The nuclei were stained with DAPI. Reference bar: 50 μm; indicated by bar in lowest right section (ProteoStat+DAPI & PES).

FIGS. 4A-4B show the accumulation kinetics of protein aggregates in ADTs treated with sera from women with early preeclampsia sera (e-PES) (FIG. 4A) and late preeclampsia sera (1-PES) (FIG. 4B) each with their respective controls. Cells were grown in media supplemented with 10% of e-PES (n=33)/control (n=39) or 1-PES (n=33)/control (n=38), fixed at various time points and stained with ProteoStat dye. Nuclei were stained with DAPI. Reference bar: 50 μm; indicated by bar in lowest right section (FIG. 4A: e-PES & 24 h; FIG. 4B: I-PES & 24 h).

FIGS. 5A-5C show the quantification of protein aggregates in different PE serum groups using the ADT-based protein aggregation assay. Comparison of protein aggregates in sera from women with early-PE (e-PES, n=33), late-PE (1-PES, n=33) and respective control (NPS-e for e-PES, n=39; NPS-1 for 1-PES, n=38) (FIG. 5A). Receiver operating characteristic (ROC) curve analysis of the abundance of protein aggregates for prediction of e-PE (FIG. 5B) and 1-PE (FIG. 5C). Data are presented as mean±SEM and analyzed by one-way ANOVA with Bonferroni post hoc test.

FIG. 6 provides confocal microscopy images showing that protein aggregates are not accumulated in autophagy-proficient trophoblasts treated with e-PE sera (e-PES) or control (Ctrl). Cells were treated with the indicated sera, fixed at 24 h, and stained with ProteoStat dye.

The nuclei were stained with DAPI. Reference bar: 20 μm; indicated by bar in lowest right section (ProteoStat+DAPI & e-PES).

FIG. 7 provides confocal microscopy images showing that depletion of the aggregates from e-PE sera attenuates the accumulation of protein aggregates in ADTs. Cells were incubated for 24 h with e-PE sera or control sera before and after depletion of aggregates, fixed, then stained with ProteoStat dye. Images are representatives of at least 3 independent experiments. The nuclei were stained with DAPI. Reference bar: 50 μm; indicated by bar in uppermost right section (ProteoStat+DAPI & ePES-filtered).

FIGS. 8A-8D demonstrate the identification of the protein components of the aggregates in PE serum using co-localization staining with specific antibodies and ProteoStat dye, and immunoblotting. ADTs were incubated with sera from e-PE (e-PES), 1-PE (1-PES) or their corresponding normal pregnancy controls and fixed at 24 h. Fixed cells were serially stained with anti-TTR (FIG. 8A) or anti-Aβ (FIG. 8B) antibodies and ProteoStat dye. The nuclei were stained with DAPI. Quantitative analysis of signal intensity of TTR and Aβ in NPS- and e-PES/1-PES-treated cells (FIG. 8C). Western blot showing separation of protein extracts under native conditions (FIG. 8D). The blots were probed for TTR and AP. Actin was used as a loading control. Experiments were repeated at least 3 times. Reference bar: 50 μm; indicated by bar in uppermost right section (Merged+DAPI & ePES).

FIGS. 9A-9B demonstrate the identification of TTR and Aβ as the components of the aggregates in ADT exposed to sera from 1-PE (1-PES). ADTs were incubated with 1-PES or control sera (Ctrl), fixed at 24 h, and immunostained for TTR (FIG. 9A) or Aβ (FIG. 9B) and counter-stained with ProteoStat dye. The nuclei were stained with DAPI. Images are representatives of at least 3 independent experiments. Reference bar: 50 μm; indicated by bar in uppermost right section (Merged+DAPI & 1-PES).

FIGS. 10A-10B provide confocal microscopy images showing that aggregated α-synuclein and SERPINA1 are not detected in e-PE serum-treated ADT. ADT were incubated with e-PE sera (e-PES) or control sera (NPS), fixed at 24 h, immunostained for α-synuclein (FIG. 10A) or SERPINA1 (FIG. 10B), and co-stained with ProteoStat dye. The nuclei were stained with DAPI. Images are representative of at least 3 independent experiments. Reference bar: 20 μm; indicated by bar in uppermost right section (Merged+DAPI & e-PES).

FIGS. 11A-11B provide receiver operating characteristic (ROC) curve analyses of TTR (FIG. 11A) and Aβ (FIG. 11B) aggregates in sera from patients with e-PE and 1-PE. Dual immunostaining for TTR and Aβ in combination with ProteoStat staining was performed in ADT cells incubated with e-PE sera (e-PES), 1-PE sera (1-PES) or respective control sera. The intensity of TTR and Aβ immunoreactive signals that were co-localized with ProteoStat fluorescence were measured and plotted for the ROC curve.

FIGS. 12A-12B show the detection of protein aggregates in sera from patients with mild cognitive impairment (MCI) and Alzheimer's disease (AD). ADTs were incubated with sera from AD patients (n=10) (FIG. 12A), MCI patients (n=14) (FIG. 12B), or their corresponding age-matched controls (Ctrl) (n=10 for AD, n=9 for MCI), fixed at 24 h, then stained with ProteoStat dye. The nuclei were stained with DAPI. Data are expressed as mean±SEM and analyzed by a student t-test. Reference bar: 50 μm; indicated by bar in each quadrant.

FIGS. 13A-13B show the detection of protein aggregates in sera from patients with mild cognitive impairment (MCI) and Alzheimer's disease (AD). The fluorescence intensity in cells were measured and statistically compared among groups; Data are expressed as mean±SEM and analyzed by one-way ANOVA (FIG. 12A). ROC analysis show robust difference in ProteoStat signal between the samples from MCI (n=14) and AD (n=10) patients (FIG. 13B).

FIGS. 14A-14B provide confocal microscopy images showing the identification of Aβ, TTR, and α-synuclein as the components of aggregates accumulated in ADTs treated with sera from AD or MCI. ADTs were treated with 10% sera from AD (n=10) (FIG. 14A), MCI (n=14) (FIG. 14B), or control (n=19), fixed at 24 h, immunostained with antibodies against Aβ, TTR, or α-synuclein, then co-stained with ProteoStat dye. Representative images are provided herein. The nuclei were stained with DAPI. Reference bars: 50 μm; indicated by bar in the following sections: Merge+DAPI & AD sections (FIG. 14A); Merge+DAPI & MCI sections (FIG. 14B).

FIG. 15 provides confocal microscopy images showing detection of phosphorylated Tau231 (T231) in sera from patients with AD and MCI. ADTs were incubated for 24 h with 10% of AD (n=4), MCI (n=4), or control sera (n=4), and immunostained with specific anti-phosphorylated T231 antibody and co-stained with ProteoStat dye. Images are representatives of at least 3 independent experiments. Reference bar: 50 μm; indicated by bar in Merge+DAPI & AD and Merge+DAPI & MCI sections.

FIGS. 16A-16B provide a comparison of the levels of α-synuclein, Aβ, and TTR aggregates in ADTs treated with serum samples from patients with MCI or AD, or serum samples from their corresponding controls. The fluorescence intensity of TTR, Aβ, and aα-synuclein in observed in FIGS. 12A-12B and FIGS. 14A-14B was measured and statistically compared among groups (FIG. 16A). ROC analyses show robust differences in TTR, Aβ, and α-synuclein between samples from MCI (n=14) and AD (n=10) patients (FIG. 16B). Data are expressed as mean±SEM and analyzed by one-way ANOVA.

FIG. 17 provides a bar graph showing a time-course study of mRNA levels of indicated proteins in ADTs exposed to sera from AD or e-PE patients. ADTs were incubated with ADS or e-PES, and total RNA was isolated at indicated time points and RT-PCR was performed. The mRNA levels were normalized to house-keeping gene, GAPDH. The ratio of the mRNA levels in ADS- or e-PES-treated cells to those in respective control serum-treated cells was calculated to reflect the transcriptional alteration of each protein at each time point, then statistically compared among the groups. Data are expressed as mean±SEM and analyzed by one-way ANOVA (n=12). ns: not statistically significant.

FIG. 18 illustrates the experimental design utilized in the mouse studies regarding the effects of trehalose on preeclampsia serum-induced early onset of AD-like features.

FIGS. 19A-19B show that preeclampsia serum induced early brain pathological changes at 4 months of age in hTAU mice. 10-12 week old non-pregnant female hTAU mice were injected with e-PE serum (100 μL/week for 4 weeks) followed by treatment (i.p injection) with either saline (100 μL /week) or Trehalose (2 g/kg/week) for 4 weeks, followed by 2 weeks of resting period prior to biological assessments. Medial prefrontal cortex (mPFC) was stained for hyperphosphorylated Tau (using antibody AT180 which detects Tau hyperphosphorylation at T231 & S235). FIG: 19A: Confocal images stained with AT180 are shown that correspond to the mPFC of control (upper), e-PES (middle), and e-PES+Trehalose (bottom), with quantification data in mPFC being present at right panels; inset images are the high magnification image of the selected area; FIG. 19B: provides a bar graph showing the number of AT180 positive cells/0.1 mm² in hTAU control, PES 49, and PES 49+trehalose treated mice.

FIGS. 20A-20B show that preeclampsia serum induced early brain pathological changes at 6 months of age in hTAU mice. 10-12 weeks old non-pregnant female hTAU mice were injected with e-PE serum (100 μL/week for 4 weeks) followed by treatment (i.p injection) with either saline (100 μL/week) or Trehalose (2 g/kg/week) for 4 weeks, followed by 8-10 weeks of resting period prior to biological assessment. Medial prefrontal cortex (mPFC) was stained for hyperphosphorylated Tau (using antibody AT180 which detects Tau hyperphosphorylation at T231 & S235). FIG. 20A: Confocal images stained with AT180 are shown that correspond to the mPFC of control (upper), e-PES (middle), and e-PES+Trehalose (bottom), with quantification data in mPFC being present at right panels; inset images are the high magnification image of the selected area. FIG. 20B: provides a bar graph showing the number of AT180 positive cells/0.1 mm² in hTAU control, PES 49, and PES 49+trehalose treated mice.

FIGS. 21A-21E provide the results of nesting studies showing nests built by hTAU mice; nests were constructed by 4- and 6-month old non-pregnant female control hTAU mice or mice injected with e-PE serum or hTAU mice injected first with e-PE serum followed by trehalose; nests were scored on a 0-7 scale; e-PES injected hTAU mice constructed significantly worse nests compared to other groups. FIG. 21A: provides photographs of representative nests constructed by the mice; FIG. 21B and FIG. 21D provide bar graphs showing nestlet score for 4-month old (FIG. 21B) and 6-month old (FIG. 21D) mice; FIG. 21C and FIG. 21E provide bar graphs showing untorn nestlet weight for 4-month old (FIG. 21C) and 6-month old (FIG. 21E) mice.

FIGS. 22A-22C provide the results of sensory-motor coordination studies utilizing a ledge assay. hTAU mice were compared to hTAU mice injected with e-PES at 10-12 weeks age (n=5) with or without trehalose administration. FIG. 22A provides photographs of hTAU control mice (mobile), e-PES only mice (immobile, falling off), and e-PES+trehalose mice (immobile, not falling off); FIG. 22B provides a bar graph with ledge scores for each subset of mice (i.e., hTAU control, PES 49, and PES 49 +trehalose). FIG. 22C provides a bar graph comparing time spent on the ledge (seconds) for each subset of mice (i.e., hTAU control, PES 49, and PES 49+trehalose).

### DETAILED DESCRIPTION OF THE DISCLOSURE

The present disclosure relates in part to an innovative blood test for the detection of proteinopathies, including AD, MCI, and PE, that does not require pre-analytic manipulation of serum and that detects total protein aggregate burden coupled with co-localization of individual biomarker proteins.

Disease progression from preclinical to prodromal and dementia stages in neurodegenerative diseases is associated with accumulation of misfolded and aggregated proteins. In the case of AD and MCI, recent studies have focused on detection of individual blood biomarkers, including Aβ and phosphorylated tau proteins (pTau181 and pTau217), and on correlation of these findings with CSF pTau181 and increased tau PET and amyloid PET uptake. These observations are important in that they can correlate specific increases in phosphorylated tau proteins with the AD continuum. Similarly, the plasma Aβ42/Aβ40 ratio is considered to be an important marker for longitudinal progression to AD dementia. Recently, α-syn has also been shown to be part of the AD pathology. While surpassing previous efforts to develop a blood test for AD, these newer proteomic tests do not detect aggregated proteins as part of a composite structure of biomarkers. To date, a rapid, sensitive, high through-put and cost-effective assay that can directly detect protein aggregates in fluids has been lacking.

It has been demonstrated that PE is a proteinopathy disease, and several aggregated proteins have been detected in urine from women with severe PE using dot-blotting in combination with Congo Red staining. Furthermore, using proteomics, immunohistochemical analysis, and western blotting, it has been demonstrated that the placenta and serum of subjects affected by PE contain protein aggregates comprising TTR. However, since acute kidney injury usually occurs in PE patients, it is not clear whether protein aggregates come directly from the injured kidneys or from the placenta through circulation.

During normal pregnancy, there is dynamic active transport of nutrients and protein molecules through the placental barrier at the maternal and fetal interface. Trophoblasts, as the frontline of the placental barrier that directly contact maternal blood, have the ability to internalize nutrients and large-sized protein molecules. The activity of the autophagy-lysosomal machinery is an essential biological process that degrades bulk proteins, such as misfolded protein aggregates or damaged organelles, to maintain cellular homeostasis.

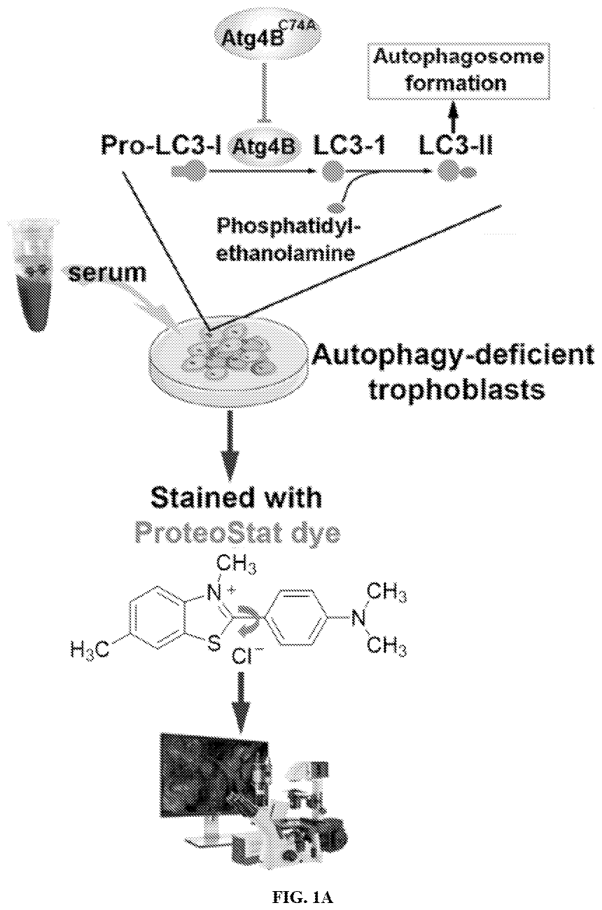

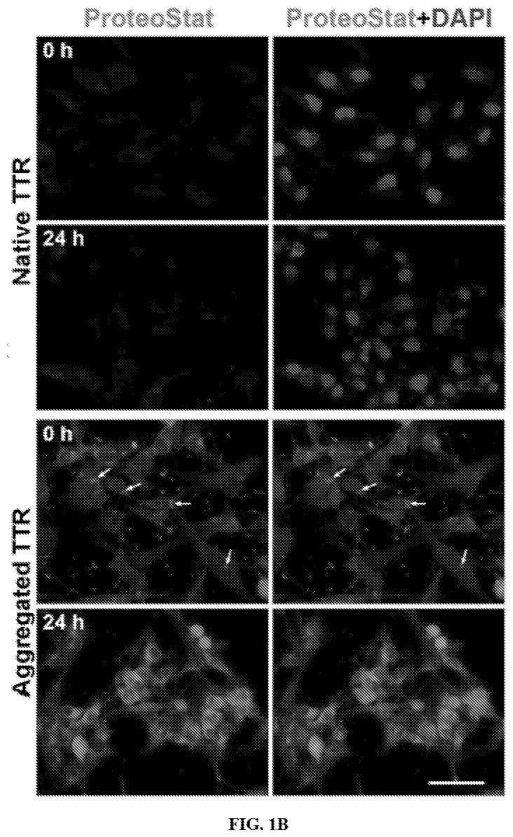

It has been shown that the accumulation of protein aggregates in the trophoblast layer of the PE placenta is a result of an impaired autophagy-lysosomal pathway. Thus, autophagy-deficient cells may accumulate protein aggregates when exposed to serum containing such complexes, and these aggregates may be detected by immunofluorescence, representing a novel strategy for the detection of protein aggregates in sera with the aid of an engineered ADT (FIG. 1).

While blood is a highly convenient fluid for a routine, non-invasive biochemical diagnosis for many diseases, it is also a very complicated fluid containing high concentrations of albumin and immunoglobulin. These plasma proteins can easily form dense structures and may mask or interfere with protein aggregates having an extremely low concentration in the serum. Therefore, a challenging issue for detection of serum protein aggregates is the requirement of high sensitivity for detection.

Previous blood-based approaches need a high volume of plasma samples (e.g. approximately 4 mL) to enrich or amplify target proteins through multiple complicated steps and expensive apparatuses, and only detect a single target protein. In contrast, the method of the present disclosure is based on detection of total protein aggregates at a cellular level using ADTs, thereby requiring minimal serum volume (e.g. 50 μL) for accurate analysis.

Moreover, the method of the present disclosure relies upon routine laboratory equipment, including cell culture systems and confocal fluorescent microscopy, to enable the identification of multiple specific proteins (e.g. TTR, Aβ, and α-syn) present in an aggregate in one assay.

Employing the method described herein, TTR, tau231, Cis P-tau, and α-syn have been identified as constituents of the aggregates observed in ADTs exposed to sera from patients with AD and MCI. Furthermore, the present disclosure provides the first evidence that TTR may exist as part of an aggregate complex together with tau, Aβ and a-syn in sera from subjects with MCI and/or AD. As a transient condition which precedes AD, no biochemical test is currently available for the diagnosis of MCI. However, the present disclosure provides evidence that aggregated serum proteins may be used as biomarkers for MCI. Thus, the method of the present disclosure may be used to predict the ones position in the AD continuum.

The reliability of the method described herein depends on whether the protein aggregates that accumulate in the ADT are internalized directly from sera or induced by pathological factors present in sera. The results of the present disclosure indicate that the accumulated protein aggregates in ADTs are mainly derived from serum. It has been demonstrated herein that ADTs are sufficiently able to internalize and accumulate in vitro generated TTR aggregates. Furthermore, depletion of aggregates from the serum remarkably attenuated the accumulation of protein aggregates in ADTs, and the accumulation of protein aggregates in ADTs occurs in a time-dependent manner. Finally, no significant transcriptional alteration of TTR, α-syn, and/or APP was observed in PE or AD serum-exposed ADTs, and α-syn only accumulated in ADTs when exposed to serum from AD and MCI patients, not PE women.

The development of a sensitive, reliable and generic protein aggregate detection assay for the early diagnosis of proteinopathy diseases is highly challenging. The present disclosure provides support for a novel, simple, highly sensitive, and cost-effective biomarker test for the detection total protein aggregates and individual proteins in serum at a cellular level. The method described herein may be of great value for the development of a point-of-care assay for the detection of serum-based protein aggregates for proteinopathy diseases, including AD, MCI, and PE, as well as the development of methods for treating, preventing, and/or ameliorating such proteinopathies.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, and molecular biology are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “autophagy-deficient” as used herein refers to a cellular state in which the natural, regulated mechanism of the cell to remove unnecessary or dysfunctional components, non-limiting examples including protein aggregates, via autophagy is substantially reduced or eliminated.

The terms “biomarker” or “marker” as used herein refers to a molecular that can be detected. Therefore, a biomarker according to the present invention includes, but is not limited to, a nucleic acid, a polypeptide, a carbohydrate, a lipid, an inorganic molecule, or an organic molecule, each of which may vary widely in size and properties. A “biomarker” can be a bodily substance relating to a bodily condition or disease. A “biomarker” can be detected using any means known in the art or by a previously unknown means that becomes apparent upon consideration of the marker by the skilled artisan.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, a “disease” is a state of health of a subject wherein the subject cannot maintain homeostasis, and wherein if the disease is not ameliorated then the subject's health continues to deteriorate.

The term “dementia” as used herein, refers to a loss of cognitive function sufficiently significant to interfere with the daily activities of a subject.

The terms “Lewy body dementia” or “LBD” as used herein refers to any manifestation of clinical symptoms attributed to Parkinson's disease dementia (PDD) and/or dementia with Lewy bodies (DLB). PDD refers dementia which has developed in a subject with established Parkinson's disease, whereas DLB refers to degenerative neurological diseases marked by motor- and non-motor related symptoms, including dementia, that is associated with the presence of Lewy bodies in the brain.

The term “mild cognitive impairment” or “MCI” as used herein refers to a subtle but measurable memory deficiency disorder which is often characterized by ongoing memory problems, but not by confusion, attention problems, or language difficulties. In certain instances, MCI may comprise a prodromal stage of Alzheimer's disease (AD).

The terms “obtaining” or “obtaining trehalose or a derivative or prodrug thereof” as used herein refers to synthesizing, purchasing, or otherwise acquiring trehalose or a derivative or prodrug thereof.

The terms “Parkinson's disease” or “PD” as used herein refers to a degenerative neurological disease marked by a combination of inhibitory motor- and non-motor symptoms, including dementia, that is associated with the presence of Lewy bodies in the brain. A subject suffering from PD may or may not present symptoms of dementia.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates), and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

The term “prodrug” as used herein refers to a derivatized form of a drug molecule that, while in certain embodiments may not be pharmacologically active itself, is chemically or enzymatically altered in the body to produce one or more active forms of the drug. A prodrug can in other embodiments be pharmacologically active, but can be enzymatically altered in the body to produce a more active form or a form with different pharmacological activity.

The terms “proteinopathy” or “proteopathy” used herein refer to any disease or disorder which is caused by the abnormal synthesis, folding, post-translational modification, or deposition of a protein in cells or tissue, which thereby disrupts the normal function of the cells, tissues, and/or organs of an affected individual. Non-limiting examples of proteinopathies include Alzheimer's disease (AD), mild cognitive impairment (MCI), and preeclampsia (PE).

The term “protein aggregate” as used herein refers to any disordered accumulation of proteins comprising at least one misfolded protein.

The term “protein aggregate dye” as used herein refers to a chemical species (e.g., small molecule) which exhibits enhanced fluorescence (e.g., red shift of fluorescence emission spectrum) upon binding to a protein aggregate. In certain embodiments, the protein aggregate comprises one or more beta sheet-rich protein structure(s), such as those found in amyloid aggregates. In certain embodiments, ProteoStat dye is a protein aggregate dye.

The term “ProteoStat dye” as used herein refers to 2-(4-(dimethylamino)phenyl)-3,6-dimethylbenzo[d]thiazol-3-ium (Thioflavin T).

As used herein, the terms “subject” and “individual” and “patient” can be used interchangeably, and may refer to a human or non-human mammal or a bird. Non-human mammals include, for example, livestock and pets, such as ovine, bovine, porcine, canine, feline and murine mammals. In certain embodiments, the subject is human.

The term “tau” as used herein refers to a protein that is the product of alternative splicing from a single gene that in humans is designated MAPT (microtubule-associated protein tau) and is located on chromosome 17.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Methods

### Methods of Detecting a Proteinopathy in a Subject

In one aspect, the present disclosure relates to a method of detecting a proteinopathy in a subject, the method comprising:
incubating autophagy-deficient trophoblast (ADT) cells in a medium comprising a serum sample collected from the subject to provide ADT cells comprising at least one protein aggregate;
staining the cells comprising at least one protein aggregate with a protein aggregate dye to provide stained cells; and
detecting the at least one protein aggregate in the stained cells.

In certain embodiments, the proteinopathy is at least one selected from the group consisting of Alzheimer's disease (AD), mild cognitive impairment (MCI), preeclampsia (PE), Lewy body dementia (LBD), gestational diabetes, and Huntington's disease (HD).

In certain embodiments, the ADT cells comprise immortalized HchEpC1b human extravillous trophoblast cells. In certain embodiments, the immortalized HchEpC1b cells are human papillomavirus E6 (HPV E6) and human telomerase reverse transcriptase (hTERT) transfected. In certain embodiments, the immortalized HchEpC1b cells are stably transfected with a mutant expression vector. In certain embodiments, the mutant expression vector is pMRX-IRES-puro-mStrawberry-Atg4BC74A. In certain embodiments, the mutant expression vector inhibits microtubule-associated proteins 1A/1B light chain 3B (MAP1LC3B-II) formation. In certain embodiments, the mutant expression vector inhibits autophagy flux. In certain embodiments, the mutant expression vector inhibits lysosomal biogenesis.

In certain embodiments, the ADT cells comprise human trophoblast TCL1 or HTR8 cells. In certain embodiments, the TCL1 or HTR8 cells are stably transfected with at least one mutant autophagy gene. In certain embodiments, the at least one mutant autophagy gene is selected from the group consisting of ATG4B, ATG7, and ATG13.

In certain embodiments, the serum sample comprises about 1, 2, 3, 4, 5, 6, 7, 8, 9, 10, 1, 12, 13, 14, 15, 16, 17, 18, 19, or about 20% (v/v) of the ADT cell incubation medium. In certain embodiments, the serum sample comprises 10% (v/v) of the ADT cell incubation medium.

In certain embodiments, the at least one protein aggregate comprises at least one protein selected from the group consisting of transthyretin (TTR), amyloid β (Aβ), α-synuclein (α-syn), Tau231 (T231), and cis P-Tau.

In certain embodiments, the staining comprises incubation of the cells comprising the at least one protein aggregate with the protein aggregate dye for a period of time selected from the group consisting of about 1, 2, 3, 4, 5, 6, 7, 8, 9, 10, 11, 12, 13, 14, 15, 16, 17, 18, 19, 20, 21, 22, 23, 24, 25, 26, 27, 28, 29, and about 30 minutes. In certain embodiments, the period of time is about 10 to about 15 minutes.

In certain embodiments, the incubation of autophagy-deficient trophoblast (ADT) cells in a medium comprising a serum sample collected from the subject provides ADT cells comprising a complex of protein aggregates. In certain embodiments, the complex of protein aggregates comprises at least one protein aggregate. In certain embodiments, the staining step comprises treating the cells comprising the at least one protein aggregate with a phosphate buffered saline (PBS) solution comprising formaldehyde to provide fixed cells. In certain embodiments, the staining step comprises incubating the fixed cells with glycine to provide quenched cells. In certain embodiments, the staining step comprises incubating the quenched cells with a permeabilizing solution to provide permeabilized cells. In certain embodiments, the staining step comprises washing the permeabilized cells to provide washed cells. In certain embodiments, the staining step comprises incubating the washed cells with the protein aggregate dye to provide stained cells.

In certain embodiments, the PBS solution comprising formaldehyde has a concentration of formaldehyde selected from the group consisting of about 1, 2, 3, 4, 5, 6, 7, 8, 9, and about 10% formaldehyde (wt %). In certain embodiments, the PBS solution comprising formaldehyde is 4% formaldehyde (wt %).

In certain embodiments, the fixed cells are incubated with glycine for a period of time selected from the group consisting of about 1, 2, 3, 4, 5, 6, 7, 8, 9, and about 10 minutes. In certain embodiments, the fixed cells are incubated with glycine for about 5 minutes.

In certain embodiments, the incubation of the quenched cells with the permeabilizing solution occurs with at least one of a period of time selected from the group consisting of about 5, 10, 15, 20, 25, 30, 35, 40, 45, 50, 55, and 60 minutes and a temperature selected from the group consisting of about −10, −9, −8, −7, −6, −5, −4, −3, −2, −1, 0, 1, 2, 3, 4, 5, 6, 7, 8, 9, and about 10° C. In certain embodiments, the incubation of the quenched cells with the permeabilizing solution occurs with at least one of a period of about 30 minutes and a temperature of 0° C.

In certain embodiments, the permeabilizing solution comprises at least one of Triton X-100, wherein the Triton X-100 has a concentration selected from the group consisting of about 0.1, 0.2, 0.3, 0.4, 0.5, 0.6, 0.7, 0.8, 0.9, and about 1.0%, and EDTA in PBS, wherein the EDTA has a concentration in PBS selected from the group consisting of about 1, 2, 3, 4, 5, 6, 7, 8, 9, and 10 mM. In certain embodiments, the permeabilizing solution comprises at least one of 0.5% Triton X-100 and 3 mM EDTA in PBS.

In certain embodiments, the permeabilized cells are washed with a PBS solution. In certain embodiments, the incubation of the washed cells occurs with at least one of: a period of time selected from the group consisting of about 5, 10, 15, 20, 25, 30, 35, 40, 45, 50, 55, and 60 minutes; an ambient temperature; and an absence of light exposure.

In certain embodiments, the protein aggregate dye is ProteoStat dye. In certain embodiments, the ProteoStat dye comprises

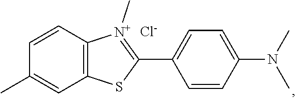

2-(4-(dimethylamino)phenyl)-3,6-dimethylbenzo[d]thiazol-3-ium (Thioflavin T).

In certain embodiments, detection of the at least one protein aggregate in the stained cells comprises measuring at least one optical property of the stained cells. In certain embodiments, the at least one optical property is fluorescence. In certain embodiments, the fluorescence is measured with a confocal microscope. In certain embodiments, the confocal microscope is equipped with a 598 red filter set. In certain embodiments, the fluorescence signal is generated using an excitation wavelength of about 550 nm. In certain embodiments, the fluorescence signal is measured with an emission filter of about 600 nm.

### Methods of Treating, Preventing, and/or Ameliorating a Proteinopathy in a Subject

In another aspect, the present disclosure relates to a method of treating, preventing, and/or ameliorating a proteinopathy in a subject in need thereof, the method comprising administering to the subject a pharmaceutical composition comprising a therapeutically effective amount of trehalose or salt, solvate, stereoisomer, derivative, prodrug and any mixtures thereof. In certain embodiments, the proteinopathy is selected from the group consisting of Alzheimer's disease (AD) and mild cognitive impairment (MCI).

In certain embodiments, the stereoisomer of trehalose is lactotrehalose.

In certain embodiments, the trehalose administration promotes degradation of at least one protein aggregate. In certain embodiments, the at least one degraded protein aggregate comprises at least one protein selected from the group consisting of transthyretin (TTR), amyloid β(Aβ), α-synuclein (α-syn), Tau231 (T231), and cis P-Tau.

In certain embodiments, the trehalose is administered to the subject daily. In certain embodiments, the trehalose is administered to the subject weekly. In certain embodiments, the trehalose is administered to the subject monthly. In certain embodiments, the trehalose is administered to the subject for a period of about one week. In certain embodiments, the trehalose is administered to the subject for a period of about two weeks. In certain embodiments, the trehalose is administered to the subject for a period of about three weeks. In certain embodiments, the trehalose is administered to the subject for a period of about one month. In certain embodiments, the trehalose is administered to the subject for a period of about two months. In certain embodiments, the therapeutically effective amount is about 1 g/kg to about 10 g/kg based on subject weight. In certain embodiments, the therapeutically effective amount is about 1 g/kg to about 5 g/kg based on subject weight. In certain embodiments, the therapeutically effective amount is about 1 g/kg to about 3 g/kg based on subject weight. In certain embodiments, the therapeutically effective amount is about 2 g/kg based on subject weight.

In certain embodiments, the pharmaceutical composition comprises trehalose and at least one pharmaceutically acceptable carrier. In certain embodiments, the subject is co-administered at least one additional agent useful for treating, preventing, and/or ameliorating a proteinopathy in a subject. In certain embodiments, the at least one additional agent useful for treating, preventing, and/or ameliorating a proteinopathy in a subject is selected from the group consisting of transthyretin (TTR) and human chorionic gonadotropin (hCG).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and assaying conditions with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### Compounds

In certain aspects of the present invention, trehalose, (2R,2′R,3 S,3′S,4S,4′S,5R,5′R,6R,6′R)-6,6′-oxybis(2-(hydroxymethyl)tetrahydro-2H-pyran-3,4,5-triol), or a salt, solvate, stereoisomer, derivative, prodrug and any mixture thereof is useful for the treatment of a proteinopathy in a subject. Trehalose is a stable disaccharide (two D-glucose molecules) having the following structure:

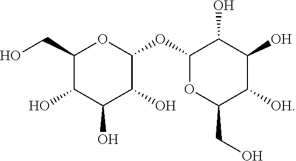 [SMILES: OC[C@H]1O[C@H](O[C@H]2O[C@H](CO)[C@@H](O)[C@H](O)[C@H]2O)[C@H](O)[C@@H](O)[C@@H]1O]

Trehalose is naturally found in over 80 organisms including yeast, bacteria, plants, fungi and invertebrates. Although trehalose has never been identified in mammals, trehalase, the enzyme that degrades trehalose, has been found in the small intestine and other organs of certain mammalian species. Trehalose is thought to protect the integrity of cells against stresses such as heat, cold, dehydration and oxidation by preventing denaturation of proteins.

Trehalose is not a substantial part of the modem diet, but is naturally present in honey, brewers and baker's yeast, mushrooms, lobster, and crab. Trehalose is generally regarded as safe for humans by the FDA based on numerous safety studies in mice, rats, and rabbits, including during pregnancy. It has been used as a preservative in the food industry, and in pharmaceuticals for the preservation of labile protein drugs. In various embodiments, the methods of the invention employ a trehalose derivative. In various embodiments, the trehalose derivative is lentztrehalose. In yet other embodiments, the trehalose derivative is trehalose hexaacetate.

In certain embodiments, the stereoisomer of trehalose is lactotrehalose, (2S,3R,4R,5S,6S)-2-(hydroxymethyl)-6-{[(2R,3R,4S,5R,6R)-3,4,5-trihydroxy-6-(hydroxymethyl)oxan-2-yl]oxy}oxane-3,4,5-triol, having the structure:

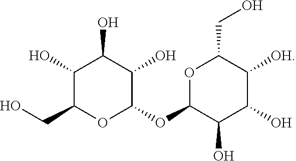 [SMILES: OC[C@@H]1O[C@@H](O[C@H]2O[C@H](CO)[C@H](O)[C@H](O)[C@H]2O)[C@@H](O)[C@H](O)[C@H]1O]

### Pharmaceutical Compositions and Formulations

The invention provides pharmaceutical compositions comprising at least one compound of the invention or a salt or solvate thereof, which are useful to practice methods of the invention. Such a pharmaceutical composition may consist of at least one compound of the invention or a salt or solvate thereof, in a form suitable for administration to a subject, or the pharmaceutical composition may comprise at least one compound of the invention or a salt or solvate thereof, and one or more pharmaceutically acceptable carriers, one or more additional ingredients, or some combination of these. At least one compound of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day. In other embodiments, the pharmaceutical compositions useful for practicing the invention may be administered to deliver a dose of between 1 ng/kg/day and 2 g/kg/day. In other embodiments, the pharmaceutical compositions useful for practicing the invention may be administered to deliver a dose of between 1 ng/kg/day and 5 g/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 mg/kg to 10 g/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the compositions and/or formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, practice the claimed methods of the present disclosure. The following working examples therefore, specifically point out the preferred embodiments of the present disclosure, and are not to be construed as limiting in any way the remainder of the disclosure.

### Materials and Methods

### Human Subjects for Preeclampsia (PE)

Patients with PE were diagnosed based on ACOG guidelines of early onset and late onset preeclampsia. Systolic blood pressures ≥140 mmHg or ≥160 mmHg and diastolic blood pressure ≥90 mmHg or ≥110 mmHg measured at or after 20 weeks of gestation were associated with PE or severe PE, respectively (Table 1). Serum samples were obtained following informed written consent from pregnant women with early onset PE (e-PE) (<34 gestational weeks), late onset PE (1-PE) (>34 gestational weeks), and gestational age-matched normal pregnancy. Exclusion criteria included chronic hypertension, gestational or pre-existing diabetes, fetal demise, daily tobacco use, fetal anomalies, and multiple gestations. For each study participant, 7-9 ml of blood was collected in BD Vacutainer SSTTM tubes and processed for serum isolation within 30 minutes. Serum samples were aliquoted in smaller volumes and stored at −80° C. until further use. Demographic and clinical data are presented in Table 1.

TABLE 1
Demographic data of patients with early-/late-
onset preeclampsia and control pregnancies

| | 1-PES | NPS-1 | e-PES | NPS-e | |
|---|---|---|---|---|---|
| Variable | (n = 33) | (n = 38) | (n = 33) | (n = 39) | p-value |
| Age (years) | 29.4 (4.8) | 29.9 (4.9) | 30.9 (5.4) | 29.6 (4.4) | NS |
| Gestational age at | 37.7 (1.8) | 37.5 (1.2) | 30.2 (2.5) | 29.9 (2.8) | NS |
| delivery (weeks) | | | | | |
| Maximum systolic blood | >160 | <120 | >160 | <120 | †<0.01 |
| pressure (mmHg) | | | | | ††<0.01 |
| Maximum diastolic | >110 | <80 | >110 | <80 | †<0.01 |
| blood pressure (mmHg) | | | | | ††<0.01 |
| Urine protein:creatinine | 3.26 (3.11) | <0.3* | 5.0 (6.6) | <0.3* | NA |

*serum creatinine, and urine protein:creatinine not mentioned in control subjects and presented as normal ranges;
†late onset preeclampsia with severe features versus term controls;
††early onset preeclampsia with severe features versus preterm controls.

### Human Subjects for Mild Cognitive Impairment (MCI) and Alzheimer's Disease (AD)

For this study, serum samples from patients with probable AD or MCI were age- and sex-matched to serum from normal controls (Table 2). Patients were evaluated and had been diagnosed with AD based on National Institute on Aging-Alzheimer's Association research criteria, or with MCI, based on research diagnostic criteria. Controls were healthy older adults, without cognitive impairment, recruited from among the clinic patients' friends and family members. Blood samples were collected and processed according to the Alzheimer's Disease Neuroimaging Initiative methods, then plasma and serum components were aliquoted into 1 mL sterile polypropylene screw capped tubes and stored frozen at −80° C. Serum samples were de-identified prior to transfer and the investigators involved in running the experiments were blind to diagnostic group until completion of lab analyses.

TABLE 2
Demographic data of patients with MCI and AD
and age-matched cognitively normal controls

| | Cognitive | | |
|---|---|---|---|
| | Normals | MCI | AD Dementia |
| | (Controls) | (Possible AD) | (Probable AD) |
| | n = 19 | n = 14 | n = 10 |
| Age, mean, yrs (SD) | 70.6 (9.3) | 70.1 (8.5) | 69.8 (9.5) |
| Sex, F/M, n | 13/6 | 8/6 | 6/4 |
| Education, mean, yrs (SD) | NA^{b} | 14.5 (2.8) | 14.3 (2.0) |
| MMSE, mean, score (SD) | NA | 26.5 (2.2) | 16.8 (7.4) |
| CDR, 0.5/1/>1, n | NA | 12/0/0 | 0/5/2 |
| AD Biomarkers^{a}, Amyloid | NA | 11/1/0 | 3/2/1 |
| PET+/CSF+/FDG-PET+, n | | | |

^{a}AD biomarker positive (+) indicates at least one of: a) amyloid plaque burden (PET brain imaging), b) cerebrospinal fluid concentrations of amyloid-beta (Aβ₁₋₄₂), total tau (T-tau), and phosphorylated tau (P-tau₁₈₁) within established reference ranges for MCI or AD dementia; or c) neuronal dysfunction measured by FDG-PET. Number of subjects (n) who underwent biomarker analysis in each category is reported;
^{b}Not assessed.

### Antibodies

The following antibodies were used in immunostaining or wester blotting: rabbit anti-TTR (A0002, Agilent Dako, Santa Clara, CA), rabbit anti-beta Amyloid 1-42 antibody [mOC98] (ab201061, abcam, Cambridge, MA), rabbit anti-α-syn (ab131508, abcam, Cambridge, MA), SERPINA1 (ARP59239 P050, Aviva Systems Biology, San Diego, CA), mouse anti-β-actin (Cell Signaling Technology), goat anti-rabbit or mouse HRP-conjugated IgG (Cell Signaling Technology), and Alexa Fluor 488 donkey anti-rabbit IgG (Molecular Probes, A-21206).

### Autophagy-Deficient Trophoblast (ADT) and Autophagy-Proficient Trophoblast (APT) Cell Lines

The HchEpC1b cell line, HPV E6 and hTERT-transfected immortalized extravillous trophoblast cells, were stably transfected with pMRX-IRES-puro-mStrawberry-Atg4BC74A, an Atg4BC74A mutant expression vector that inhibits MAP1LC3B-II formation, as previously described. For the control, HchEpC1b cells were stably transfected with pIVIRX-IRES-puro-mStrawberry, a control vector only encoding monomeric red fluorescent protein. After transfection, the cells were grown in RPMI1640 medium (GIBCO, 11875, MA, USA) supplemented with 10% FBS and selected by addition of 0.3 μg/ml puromycin (Sigma) in the medium.

### In Vitro Generation of TTR Aggregates

Native human TTR solution (1 mg/mL, AbD, Serotec) was diluted in sodium acetate buffer at pH 3.5 containing 100 mM KCl, 1 mM EDTA and 200 mM sodium acetate. A commercial kit was used to qualitatively measure protein aggregation of native and aggregated lysozyme as negative and positive controls along with BSA in this analysis. For a negative control, the same concentration of BSA solution was made with the same buffer. Both solutions were incubated in sealed tubes for 5-7 days at 37° C. In pilot experiments, protein aggregates were qualitatively verified using a protein aggregation assay kit according to manufacturer's instruction (Enzo Life Sciences). Briefly, samples were mixed with ProteoStat dye and incubated for 10-15 mins at room temperature. Fluorescence signal intensity was read with a fluorescence microplate reader (Spectra Amax GEMINIEM, Molecular Devices) using an excitation setting of about 550 nm and an emission filter of about 600 nm.

### ADT-Based Protein Aggregate Detection Assay

ADT or APT were plated on sterile glass coverslips and grown in RPMI1640 medium (GIBCO, 11875, MA, USA) supplemented with 10% FBS. After 65-70% confluency, the cells were washed with DPBS and then incubated in FBS-free RPMI1640 medium supplemented with 10% sera from women with e-PES, 1-PES, or their respective gestational age-matched controls, or sera from patients with AD, MCI or their respective age-matched controls. Treated cells were fixed at various time points with 4% formaldehyde in phosphate buffered saline (PBS) for 30 min at room temperature and then quenched with glycine for 5 mins. The cells were permeabilized for 30 min with a solution containing 0.5% Triton X-100 and 3 mM EDTA in PBS on ice. The cells were washed with PBS and then incubated with ProteoStat dye for 20 min at room temperature in darkness according to the manufacturer's instructions (Enzo Life Sciences). The cells were then washed with PBS, mounted on glass slides with anti-quench mounting medium with DAPI (Vector Laboratories, Inc., Burlingame, CA), and observed using a confocal microscope (Nikon A1R, Japan) equipped with a 598 Red filter set. All images were acquired with a 60× objective lens. The signal intensity was measured using ImageJ software (NIH). Figures were processed with brightness/contrast adjustment using Photoshop CS2 (Adobe) using the same settings.

### Identification of the Components of Protein Aggregates

Cells were treated for 24 h, fixed and permeabilized in the same manner as described elsewhere herein. The cells were blocked by using a solution containing 1% BSA, 10% normal donkey serum and 0.3 M glycine in PBS for 1 hour at room temperature followed by overnight incubation with primary antibodies against TTR, Aβ, α-syn and SERPINA1 (1:250 dilutions in blocking solution) in a humid chamber at 4° C. The cells were washed three times in PBS buffer (pH 7.4) containing 0.1% Tween 20 (PBST) and incubated with Alexa Fluor 488 secondary antibody at 1:500 dilutions in PBS containing 1% BSA for 2 h in dark. The cells were then counter-stained with ProteoStat dye (prepared according to kit instructions) for 30 min at room temperature. Finally, the slides were washed three times with PBST, mounted and observed under a confocal microscope (Nikon, A1R, Japan) using a Texas Red filter set for the ProteoStat dye and an FITC filter set for Alexa Fluor 488 conjugated antibodies. All images were acquired with a 60× objective lens. Negative controls were performed by replacing the primary antibody with purified rabbit IgG or mouse IgG. Immunoreactive intensity was measured with ImageJ (NIH). Figures were processed with brightness/contrast adjustment using Photoshop CS2 (Adobe) using the same settings.

### Quantitative Reverse Transcription PCR (qRT-PCR)

Total RNA was isolated from ADTs treated for 3 h, 12 h, and 24 h with sera from e-PE, AD, or their respective controls by using the TRIzol (Invitrogen, USA). The cDNA synthesis was carried out according to the protocol of M-MuLV Reverse transcriptase kit (MBI Fermentas, USA). qRT-PCR was performed with Power SYBR Green dye (Applied Biosystems, Foster City, CA) using the following set of primers 5′-CAAGCAGTGCAAGACCCATC-3′ (SEQ ID

NO:1) and 5′-AGAAGGGCATCACTTACAAACTC-3′ (SEQ ID NO:2), for Amyloid beta (A4) precursor protein, 5′-TGGGAGCCATTTGCCTCTG-3′ (SEQ ID NO:3) and 5′-AGCCGTGGTGGAATAGGAGTA-3′ (SEQ ID NO:4) for TTR, and 5′-TGGTGAGCGAAACAGAAGCC-3′ (SEQ ID NO:5) and 5′-TCCATAGCAACCTGCGTAATGAA-3′ (SEQ ID NO:6) for α-Syn. Step One Plus Real Time PCR System (Applied Biosystems, Foster City, CA) was used for all the quantitative qRT-PCR data at the melting temperature of the corresponding genes. All reactions were run with the following program: 95° C. for 10 min, followed by 35 cycles of 95° C. for 15 s, 55-61 ° C. for 30 s, and 72° C. for 30 s, finishing with melting curve step. The collected CT values were normalized to internal control, gapdh. The obtained values from either e-PE or AD group were compared with respect to values obtained from their respective control groups. There were five determinations using five individual experiments. The results were expressed as the fold change of corresponding genes of the control.

### Immunoblotting

To discern the conformation of protein aggregation, equal amounts of protein extracts were separated with 4-15% SDS-PAGE under native conditions. Protein extracts were mixed with the sample buffer that does not contain reducing agents and SDS; the samples were not heated and were run in SDS-free Tris Buffer (Bio-Rad). After blocking in 5% nonfat dry milk dissolved in PB ST for 1 h, the transferred PVDF membrane (Bio-Rad) was incubated overnight in primary antibody solution diluted in 5% nonfat milk or 3% BSA in PBST at 4° C. After sufficient washes, the membrane was incubated for 1 h at room temperature with HRP-conjugated donkey anti-rabbit or mouse IgG (Cell signaling), treated with chemiluminescence substrate (SuperSignal, Pierce) and exposed on film (Kodak) or imaged using ChemiDo XRS+(BIO-RAD). Density of bands was measured using ImageJ.

### Statistical Analysis

Data were presented as the mean ±SEM and comparisons between experimental groups were statistically analyzed using a Student t test or one-way ANOVA followed by a post hoc test if p value is significant (GraphPad Prism Software, Inc). Differences between the groups were considered significant when the p value was <0.05. Diagnostic accuracies were assessed with receiver operating characteristic (ROC) curve analysis using MedCalc Software version 19.3. The output included area under the curve (AUC), 95% confidence interval (CI) for the AUC, sensitivity, specificity, and significance level (p value).

### Nesting Activity Assay

Nesting activity was assessed using a modified scoring criteria to rate the quality of nest construction and the amount of torn nestlet material. Following the nocturnal period, untorn nesting material was weighed, and nests were photographed for scoring on a 0-7 ordinal scale modified from published criteria. Nestlet scores and weights are shown as mean+SEM; *p<0.05 versus controls. Preeclampsia serum was used to observe significant effects in hTAU mice without waiting for 9-12 months of age.

### Ledge Assay

Sensorimotor coordination deficits were detected by the ledge assay. In the ledge assay, mice were placed on the elevated cage's ledge at a height of 35 cm and width of 0.8 cm and their movement was monitored. Each mouse was tested three times (approximately 20 seconds) and scored from 0 to 3 depending on the severity of deficits. Scoring proceeds as follows: if the mouse walked along the ledge without food faults (i.e. losing footing) and back into the cage delicately—score of 0; if the mouse demonstrated any foot fault while walking on the ledge—score of 1; if the mouse did not effectively walk on the ledge or dismounted the ledge immediately—score of 2; if the mouse fell off of the ledge or avoided walking—score of 3.

### Example 1: Validation the Protein Aggregate Detection Strategy

A strategy was designed to detect multiple proteins and their aggregates, as a number of proteins were reasoned to be involved in the pathological progression of proteinopathy diseases. The present strategy is based on the observation that the protein aggregates accumulate in human trophoblasts when autophagy is impaired by induction of endoplasmic reticulum (ER) stress. Furthermore, prior studies have shown that trophoblast cells have strong endocytic and phagocytic ability to internalize large molecules and complexes. Thus, protein aggregate complexes are readily endocytosed, but not easily degraded or cleared by autophagy deficient trophoblasts (ADT).

Validation of the protein aggregate detection strategy was initiated by the establishment of an ADT cell line using human first trimester extravillous trophoblasts by stably transfecting and expressing ATG4BC74A, an inactive mutant autophagy gene of ATG4B. The mutant ATG4BC74A inhibits conversion of LC3-I to LC3-II and subsequently blocks autophagic flux (FIG. 1A). The engineered ADTs were confirmed to exhibit not only disrupted autophagic flux, but also impaired lysosomal biogenesis in prior studies.

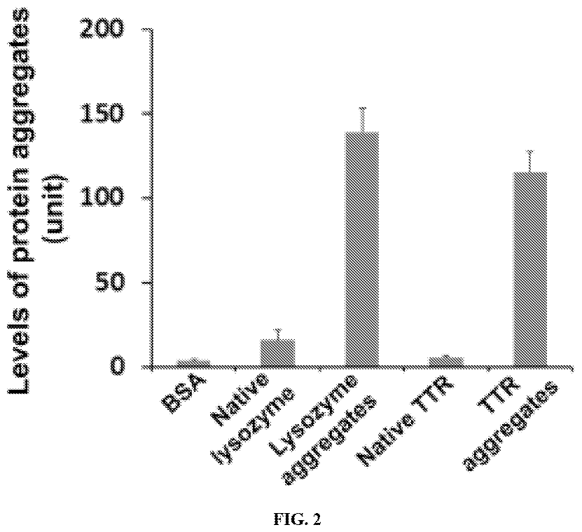

Next, the propensity of ADTs to accumulate protein aggregates was evaluated using aggregated transthyretin (TTR), which has been detected in proteinopathy diseases (e.g. preeclampsia), as an exemplary protein aggregate component. Utilizing an in vitro method, described elsewhere herein, TTR aggregates formation was induced, and the conversion of TTR into aggregates was confirmed by a rapid optimal density (OD) analysis that detects altered fluorescence readings for dye-aggregate complexes (FIG. 2).

For the cell-based assay, ADTs were incubated on ice with in vitro generated TTR aggregates to allow synchronization of entry. After 20 min incubation, the cells were either immediately fixed (referred to as 0 h time point) or incubated at 37° C. for 24 h. As controls, the cells were incubated with native (non-aggregated) TTR in parallel. The fixed cells were then stained with ProteoStat.

In ADT cells treated with aggregated TTR at 0 h, ProteoStat-positive clump-like aggregates were distributed on the surface and in extracellular areas of the cells (FIG. 1B). After 24 h incubation, robust ProteoStat fluorescent signals for TTR aggregates were readily detected in almost all cells in the well (FIG. 1B). In contrast, little to no ProteoStat signal was observed in ADT cells incubated with native TTR (FIG. 1B). Therefore, it has been demonstrated that aggregated protein complexes may accumulate in ADTs, and that these protein aggregates may be easily detected by using ProteoStat. Thus, this novel method provides a basis for detecting protein aggregates in serum and in fluids such as serum and urine.

### Example 2: Serum-Based Detection of Protein Aggregates

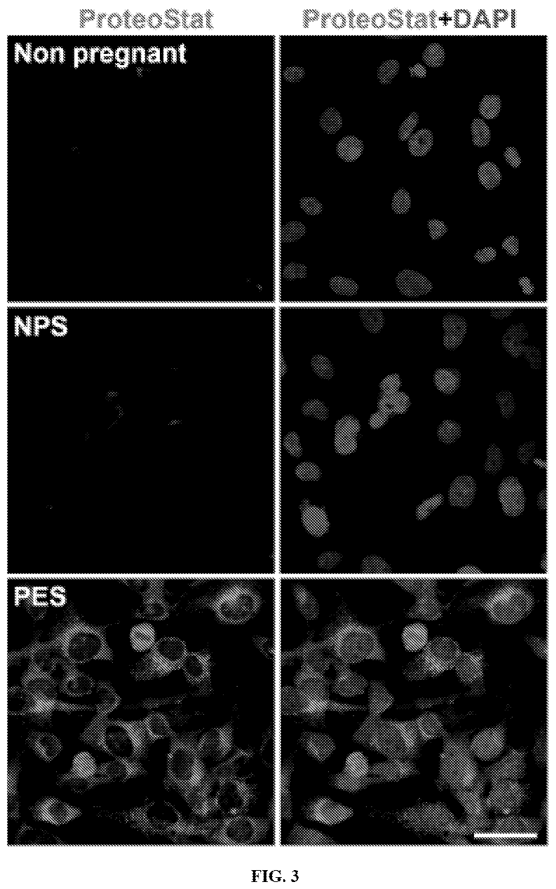

Sera from early (e-PES) and late (1-PES) onset PE patients and gestational age-matched normal pregnancy controls were tested in pilot experiments. As described elsewhere herein, PE diagnosis followed the American College of Obstetricians and Gynecologists (ACOG) guidelines (Table 1). Serum samples from non-pregnant women (n=4), as well as with women with normal pregnancy (n=4) and women with e-PE (n=4) were initially analyzed. ADT cells grown on cover slips were incubated with serum samples for 24 h. Cells were fixed and stained with ProteoStat. Cytoplasmic accumulation of protein aggregates was confirmed by ProteoStat fluorescence signal, as DAPI-stained nuclei were almost devoid of any other fluorescence signal. Data shown in FIG. 3 strongly suggest that ADTs can be used to detect serum protein aggregates from PE patients.

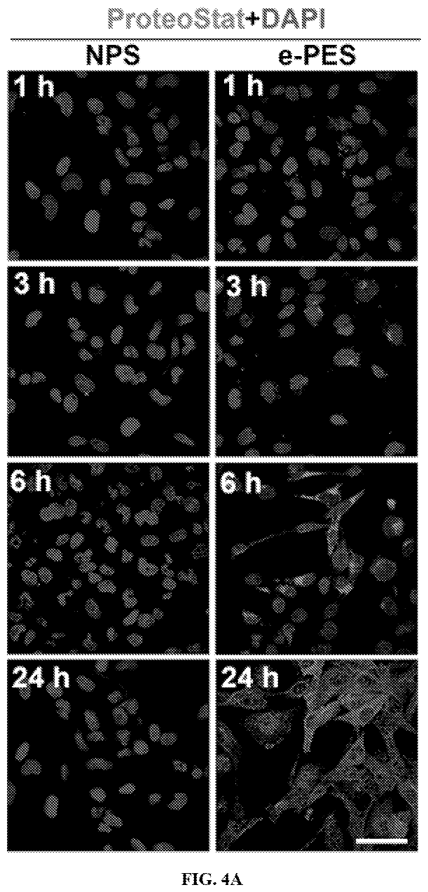

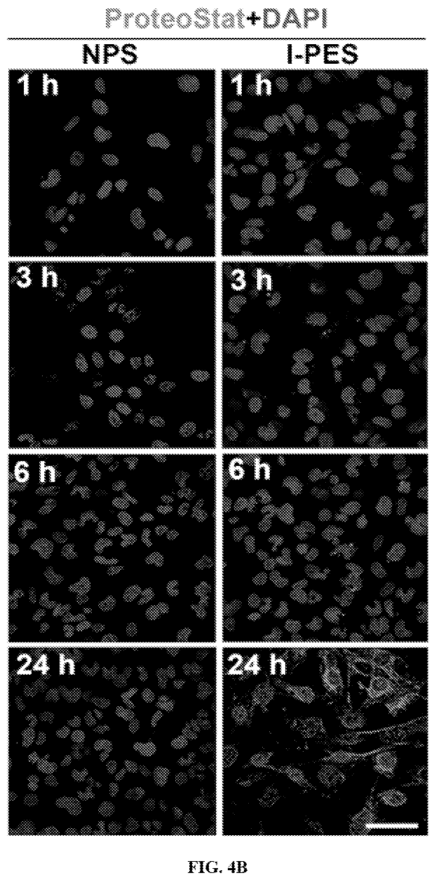

To generalize the ADT-ProteoStat protein aggregate detection assay and to assess the kinetics of accumulation of protein aggregates, ADTs were incubated with FBS-free medium supplemented with 10% sera from women with e-PE (n=33), 1-PE (n=33), or respective gestational age-matched normal pregnancy controls (n=39 for e-PE, n=38 for 1-PE) (Table 1). Cells were fixed at indicated time points and then stained with ProteoStat dye. The pixel intensity of the ProteoStat signal, as analyzed under a confocal microscope, was measured with ImageJ software and statistically compared among different groups at various time points. ProteoStat-positive protein aggregates started to appear in a few cells treated with e-PE serum (e-PES) even at 1 h (FIG. 4A).

The total number of aggregate-containing ADT cells and intensity of the ProteoStat signal increased in a time-dependent manner. At 24 h, almost all cells (>85%) displayed robust ProteoStat signal. In contrast, only a very weak signal was observed in cells exposed to sera from gestational age-matched controls even at 24 h (FIG. 4A). With 1-PE serum exposure, only a small number of cells showed fluorescent signal at 6 h, the intensity of which was lower than that with e-PES treatment at 6 h.

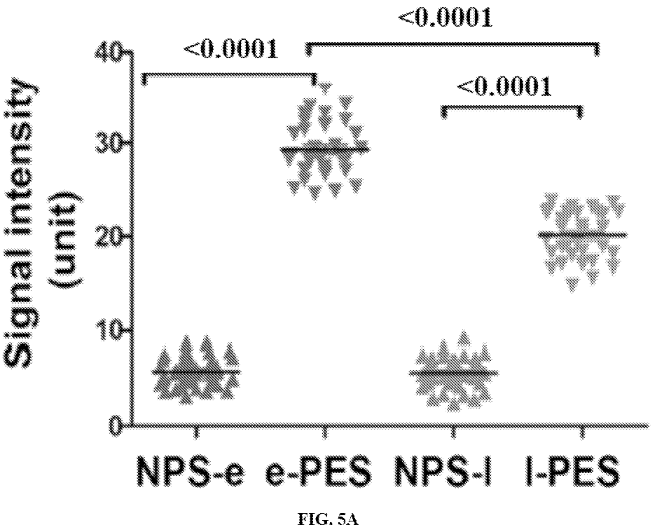

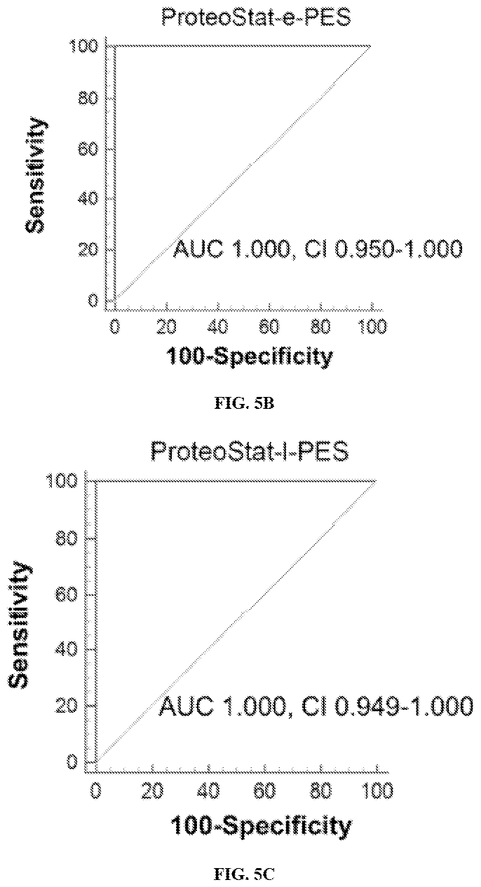

However, at 24 h, about 60% of cells exhibited relatively stronger ProteoStat signal (FIG. 4B). Quantitative analysis revealed a significant increase in the pixel intensity of the ProteoStat fluorescence signal in ADTs incubated with either e-PES or 1-PES vs. respective controls at 24 h (FIG. 5A, p<0.01). The increased abundance of protein aggregates in e-PES and 1-PES compared to control serum samples is reflected by the high Area Under Curve (AUC) on ROC analysis of 1.0, p<0.001 and CI of 0.949-1.000 (FIGS. 5B-5C). To better distinguish e-PE from 1-PE, a careful screening of a larger set of samples at earlier time points with less signal intensity may be needed.

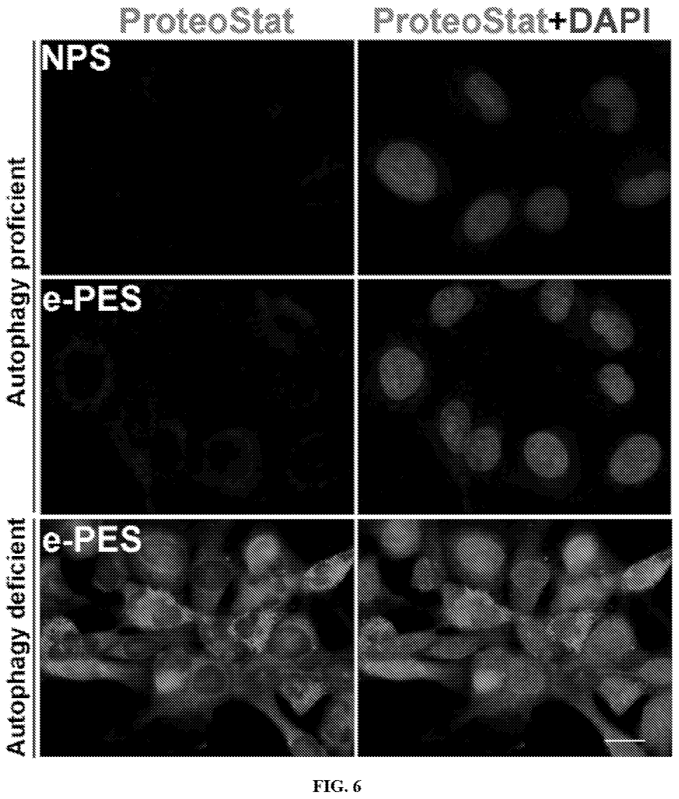

To further demonstrate the significance of ADTs for the detection of protein aggregates, autophagy-proficient trophoblasts (APTs) and ADTs were compared under identical incubation conditions for 24 h. APTs exhibited little to no ProteoStat fluorescence even after 24 h exposure to e-PES as compared to ADTs (FIG. 6).

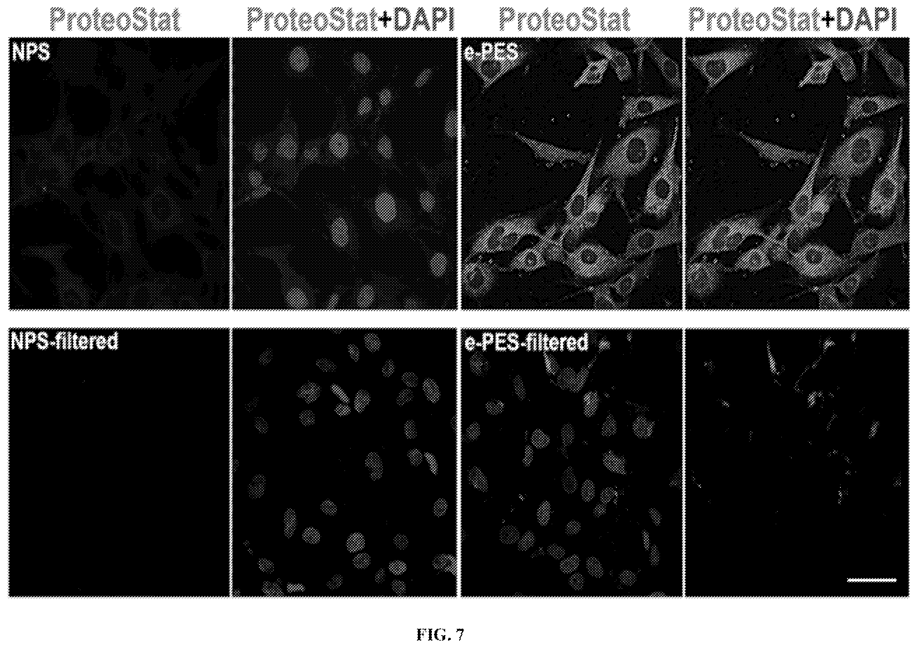

Next, it was determined whether or not the protein aggregates detected in serum-exposed ADTs were directly derived from the input PES. To address this, serum was first depleted of protein aggregates by filtration through a nitrocellulose membrane with a pore size of 0.22 μm. ADTs were then incubated with filtered e-PES or unfiltered serum sample from the same patient(s). Filtration of PES through a nitrocellulose membrane capable of trapping large-sized protein structures resulted in nearly complete depletion of protein aggregates as depicted by a very poor ProteoStat signal in ADT cells (FIG. 7), suggesting that accumulated protein aggregates detected in ADTs originated in sera from PE patients. In contrast, normal pregnancy serum (NPS) from controls showed no ProteoStat signal, supporting earlier findings reported elsewhere herein.

### Example 3: Identification of the Protein Components of the Aggregate Complex in Sera from PE Patients

To identify specific proteins in aggregate complexes in PES, co-localization staining was performed using specific antibodies in combination with the ProteoStat dye. Also of interest was the determination of whether or not PE and AD share common protein aggregate markers. Previous studies have suggested the presence of TTR aggregates in the sera from PE patients, whereas SERPINA1 may be present in the urine of subjects with severe PE. Based on previous observations on TTR and other findings, the presence of TTR, Aβ, α-synuclein (α-syn) and SERPINA1 as components of protein aggregates in PES-treated ADTs was investigated.

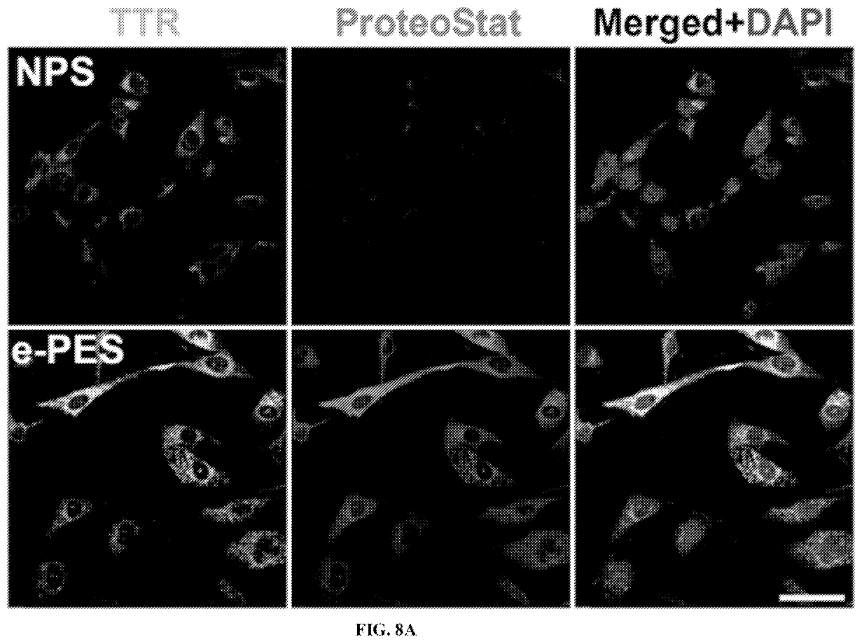

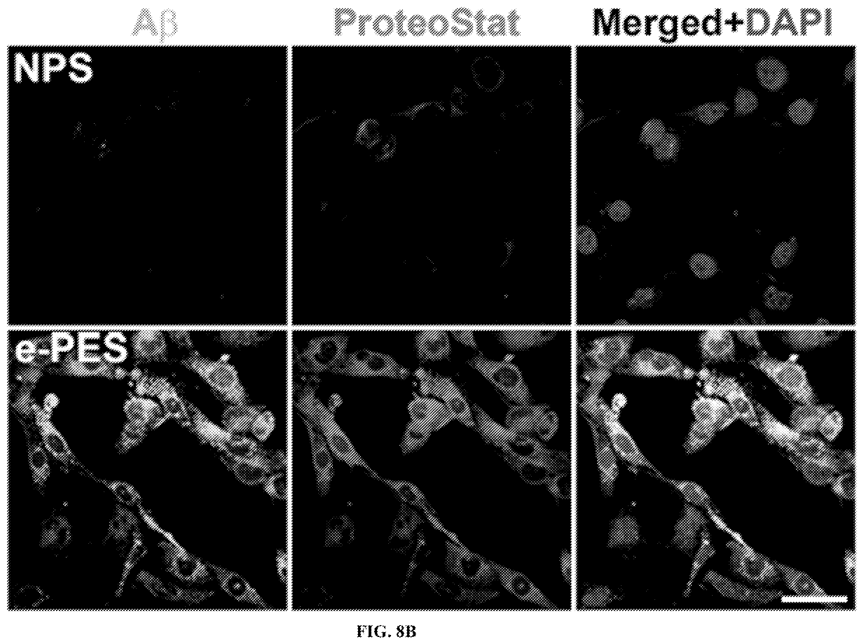

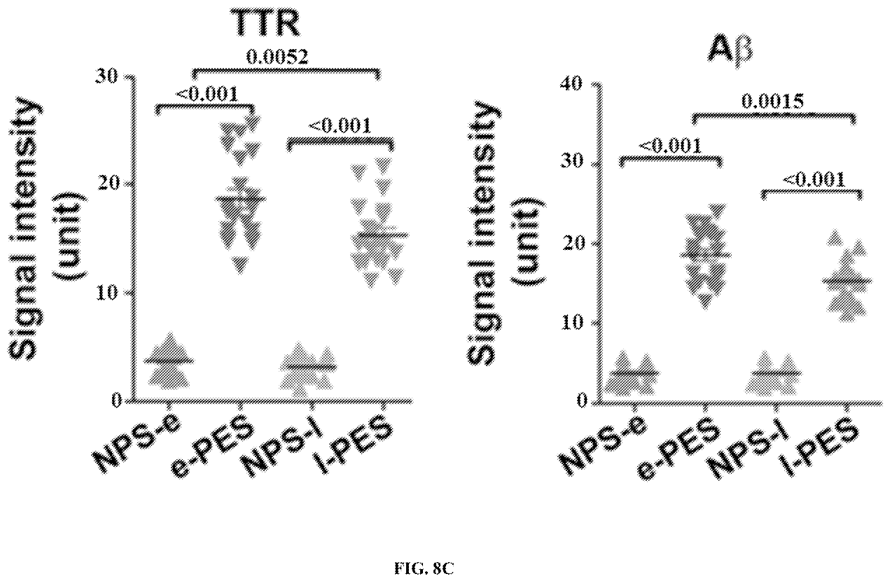

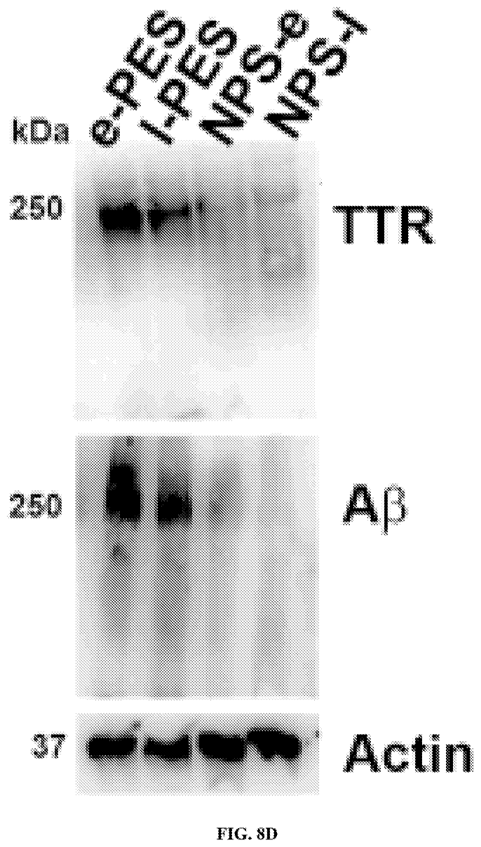

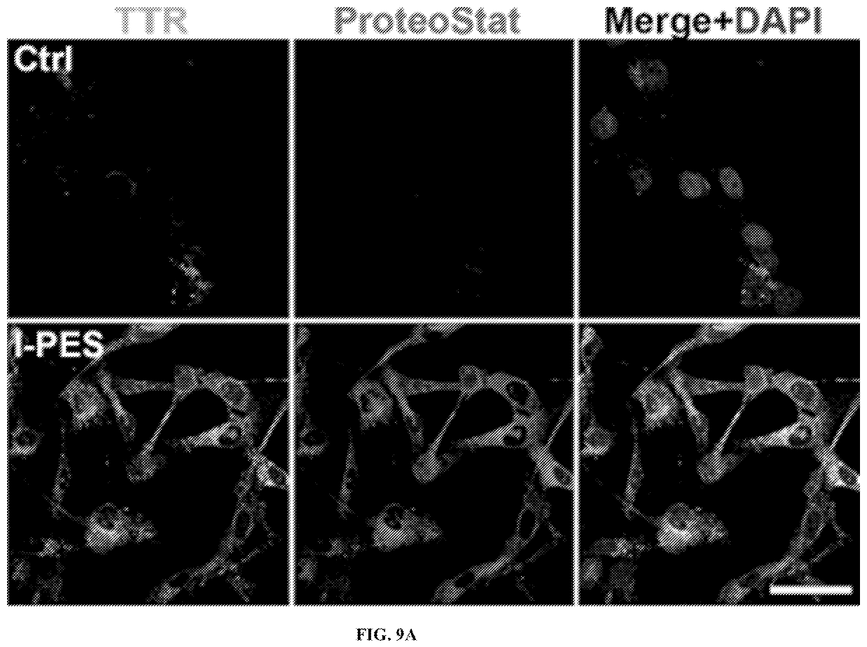

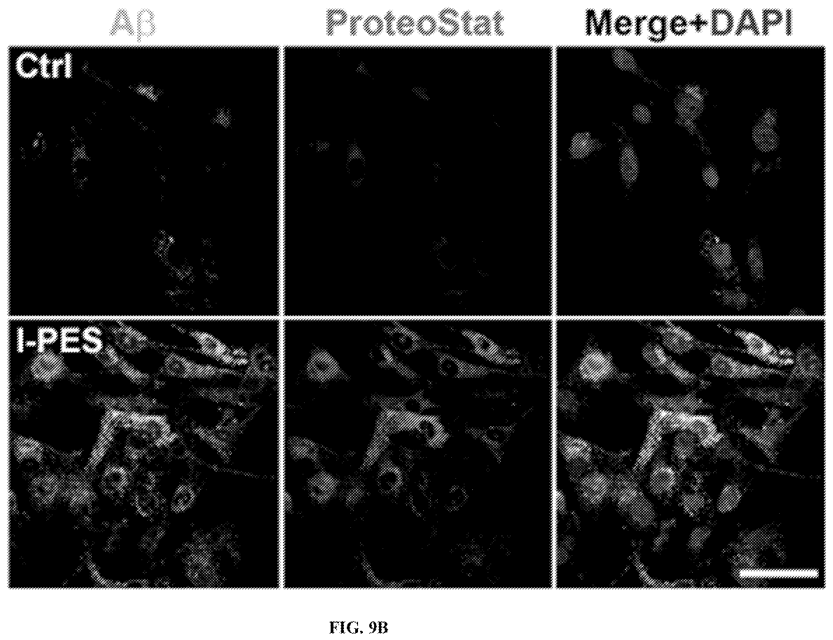

Results indicated that a large amount of TTR and Aβ, as well as robust ProteoStat fluorescence, were detected in e-PES-treated cells. Importantly, both TTR and Aβ immunoreactive signals were co-localized with the ProteoStat signal, indicative of aggregated nature of TTR and Aβ (FIGS. 8A-8B). Similar results were obtained for 1-PES (FIG. 9). By comparison, weak TTR and little to no AP ProteoStat signals were seen in cells treated with gestational age-matched control sera (FIGS. 8A-8B).

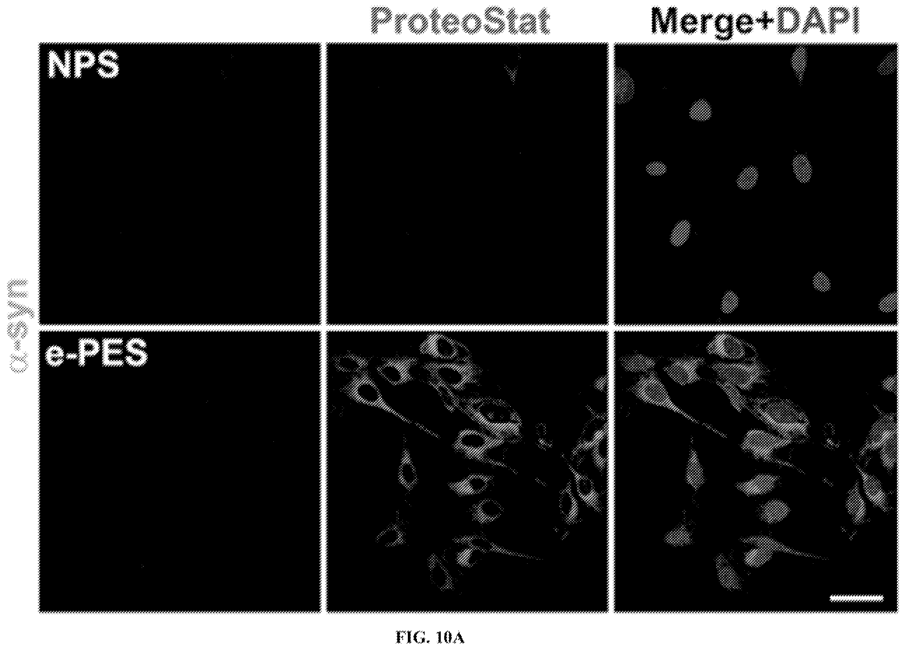

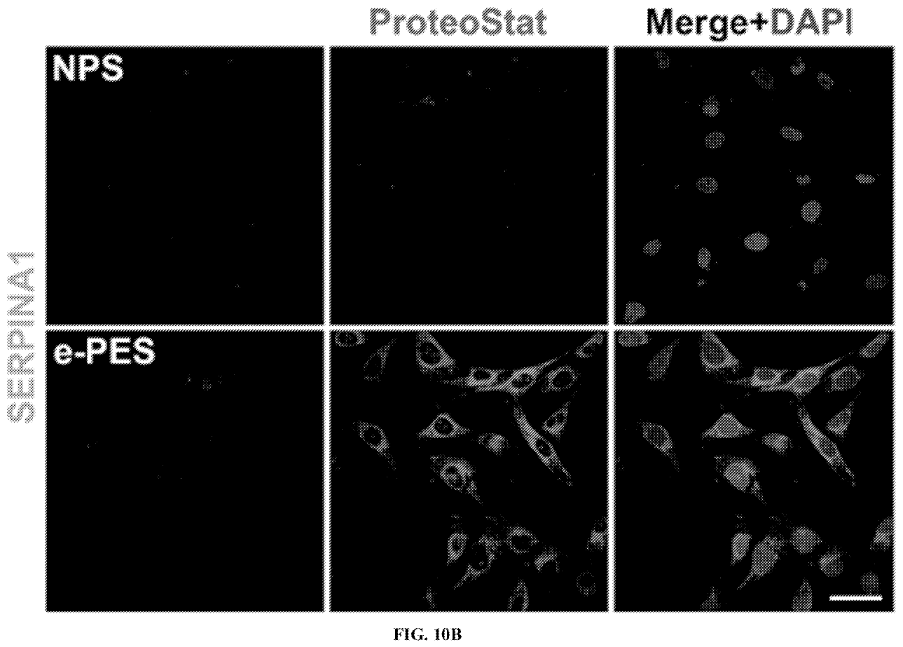

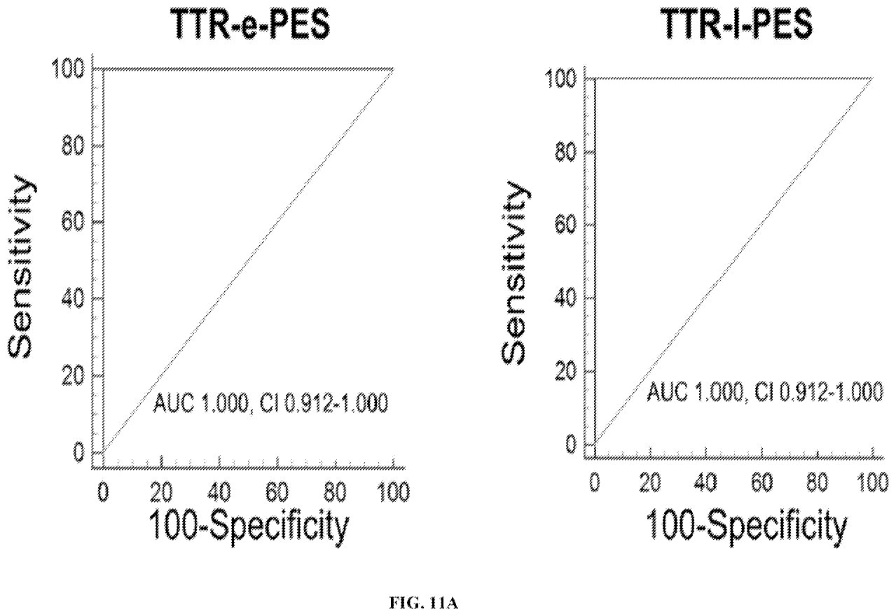

Importantly, no immunoreactive SERPINA1 or a-syn were observed in either e-PES/1-PES-treated or control serum-treated ADTs, although robust ProteoStat signal was present in e-PES/1-PES-exposed ADTs (FIG. 10). The pixel intensity of TTR and Aβ immunoreactive signals was measured with ImageJ and statistically compared among the groups. The quantitative analysis showed higher levels of TTR and Aβ in e-PES compared to 1-PES (FIG. 8C). Like the highly significant ROC curve AUC and CI values for ProteoStat analysis, these values demonstrated an AUC of 1.0 and a CI of 0.912-1.000 (FIG. 11).

To validate the findings described above, western blotting was utilized under native conditions to directly separate protein aggregates from extracts from the ADTs exposed to e-PES, 1-PES, or respective controls. A large amount of protein aggregates with high molecular weight were identified by anti-TTR and anti-Aβ antibodies in the ADTs treated with e-PES or 1-PES, but not from cells treated with corresponding control sera (FIG. 8D). These results are consistent with those obtained from the co-localization staining, indicating that TTR and Aβ, not α-syn and SERPINAL are components of aggregated protein complexes in PE sera.

### Example 4: Application of ADT-ProteoStat Assay to Sera from AD and MCI Patients

To test the applicability of this ADT-ProteoStat assay for other well-known proteinopathy diseases, we investigated whether this assay can be employed to detect protein aggregates in sera from patients with AD and MCI. Participant characteristics describing baseline demographics, clinical assessments, imaging measures, and fluid biomarker information are shown in Table 2. Dementia severity was classified according to the Clinical Dementia Rating scale (CDR), a widely used and validated clinical scale that stratifies cognitive and functional impairment into group 0 (normal), group 0.5 (very mild cognitive impairment or questionable dementia), or groups 1-3 (mild to severe dementia). Biomarker support for AD pathology was available for 12/14 MCI cases. The two other cases lacked biomarker tests as part of their clinical diagnostic evaluations. These cases were highly likely to have AD pathology, however, as previous research has shown that people with CDR 0.5 or MCI almost always have AD pathology post-mortem. Biomarker support for AD pathology was available for 6/10 probable AD cases diagnosed according to National Institute on Aging-Alzheimer's Association research criteria. The four other cases lacked biomarker tests as part of their clinical diagnostic evaluations, but these cases also conformed to the National Institute on Aging-Alzheimer's Association research criteria. The overall design was to compare group AD severity categories according to the CDR so as to avoid arbitrary distinctions.

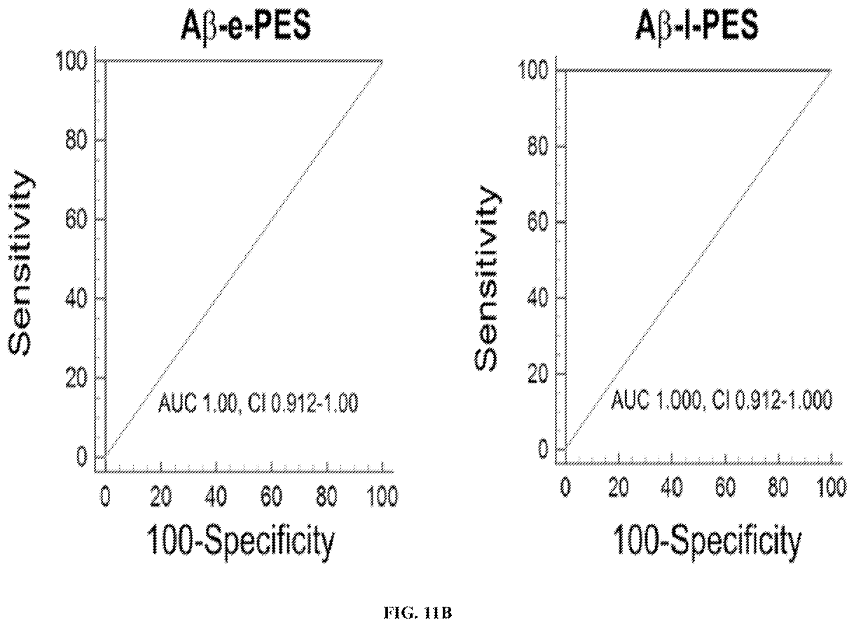

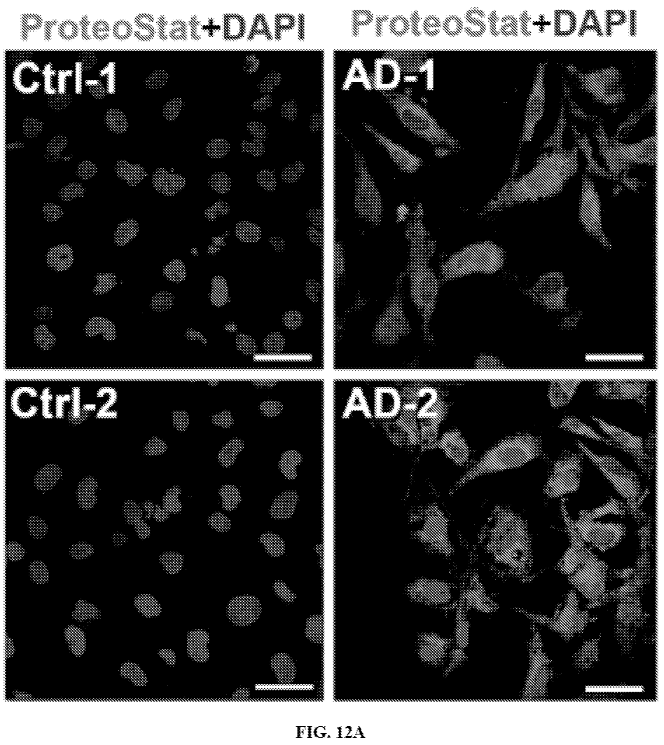

ADT cells were exposed for 24 h to FBS-free medium supplemented with serum from AD and MCI patients at 10% v/v concentration as described for PE serum samples. The results revealed a large amount of protein aggregates, as demonstrated by robust ProteoStat signals in the cells treated with serum samples from AD patients (n=10), but not age-matched control serum samples (n=19). Representative experiments are shown in FIG. 12A and FIG. 12B. ROC analysis to distinguish cases from controls and MCI from AD revealed significant AUC values of 0.832-1.00. MCI is a pre-AD condition and would be expected to show equal or less content of protein aggregates compared to those with AD dementia.

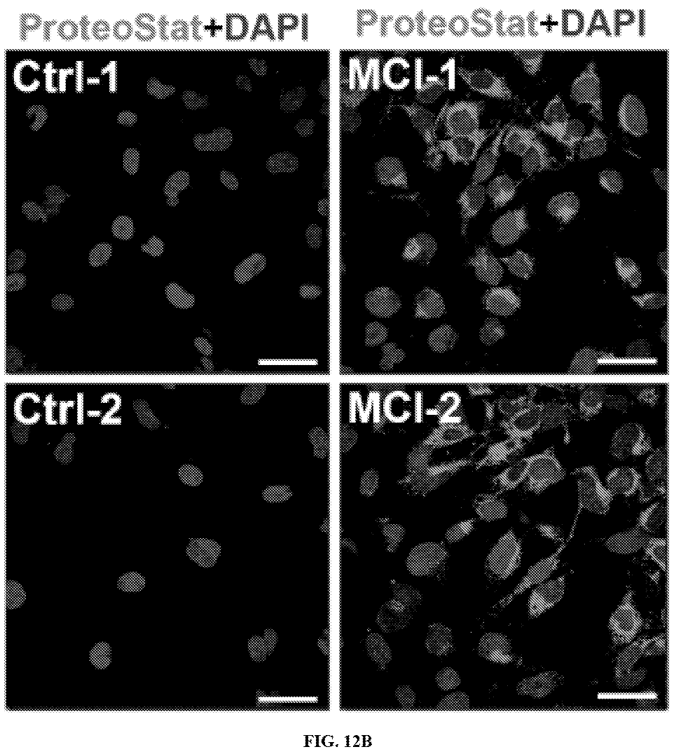

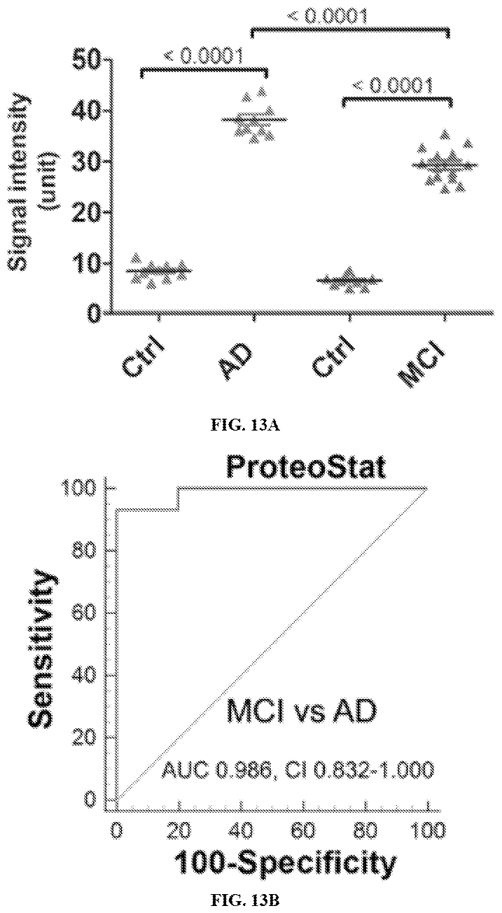

Thus, it is important to assess whether the ADT-ProteoStat assay can be applied to sera from MCI patients to detect similar protein components and content of aggregates at an earlier stage than observed in the AD serum samples. Accordingly, serum samples from MCI patients (n=14) who were diagnosed clinically, as described elsewhere herein, were tested for the purpose of detecting protein aggregates. Significant levels of aggregated proteins were detected in the ADTs treated with MCI serum relative to age-matched controls (FIG. 12A and FIG. 12B). Quantitative data indicated a significant increase in the intensity of ProteoStat fluorescence signal for detection of aggregates of TTR, Aβ, α-syn and ProteoStat in AD and MCI serum-treated ADTs and relative to respective age-matched control samples (FIG. 13A). It is important to note that while the sample size is small, because of strict definition of clinical conditions in the cases and age-matched controls, a discriminative assay was nonetheless achieved. Furthermore, for AD and MCI serum samples, the research team was blind to group assignment (patients vs controls) and the samples were decoded only after completion of detection assays.

### Example 5: Identification and Validation of the Components of Protein Aggregates in Sera from MCI and AD Patients

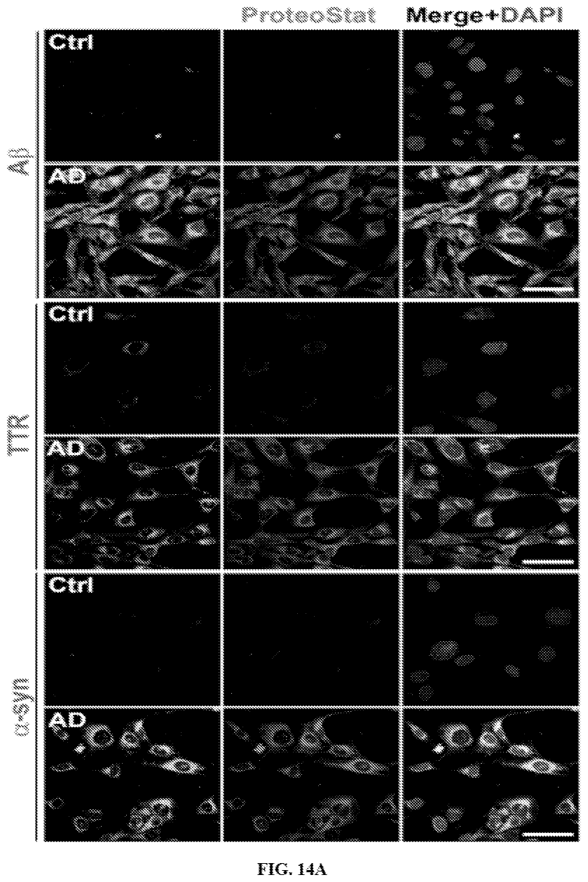

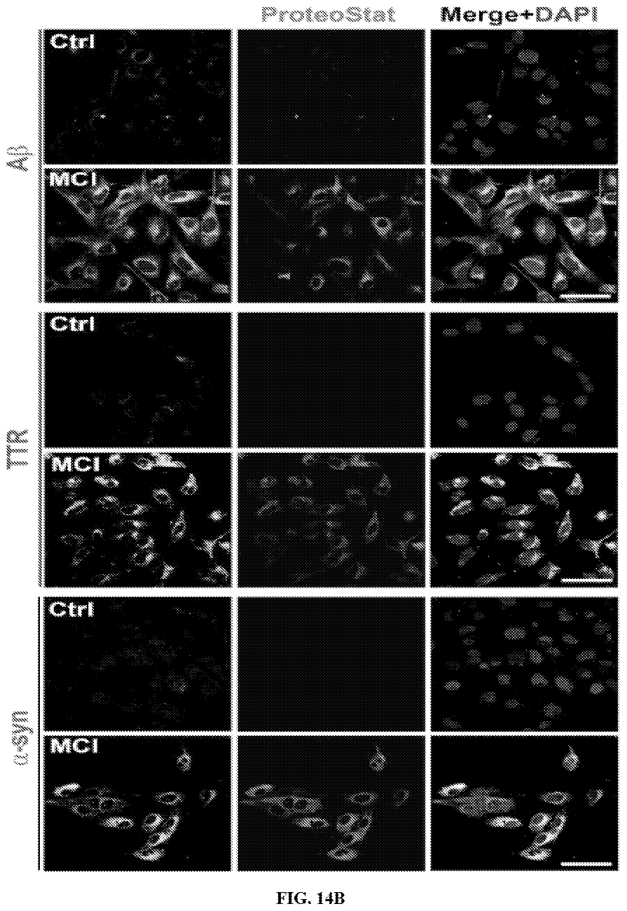

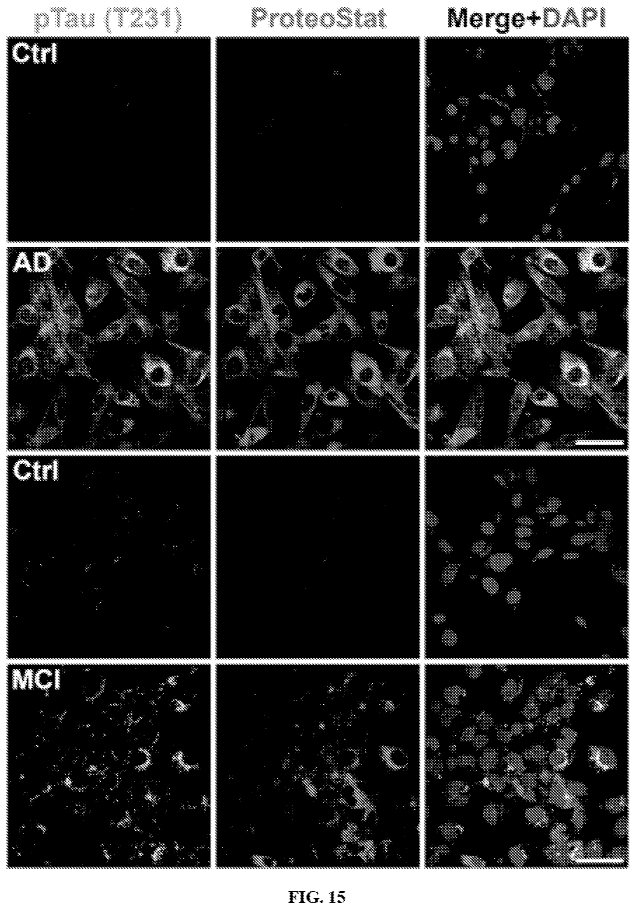

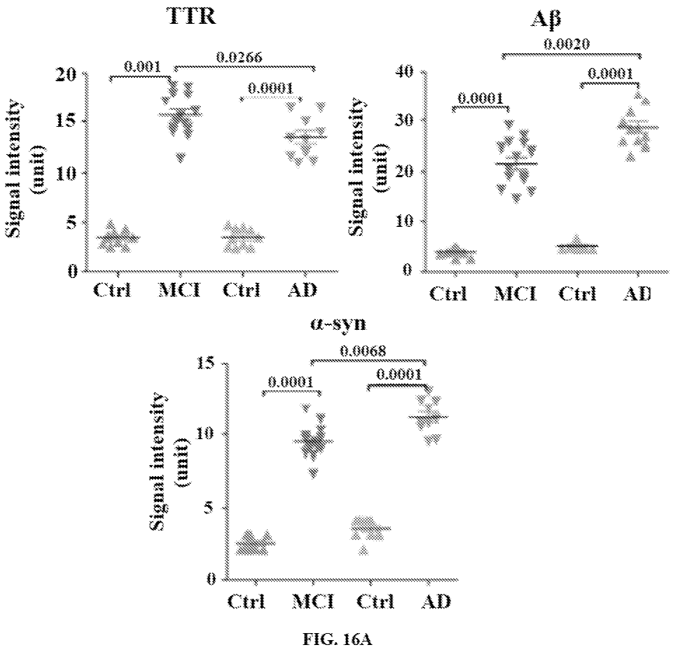

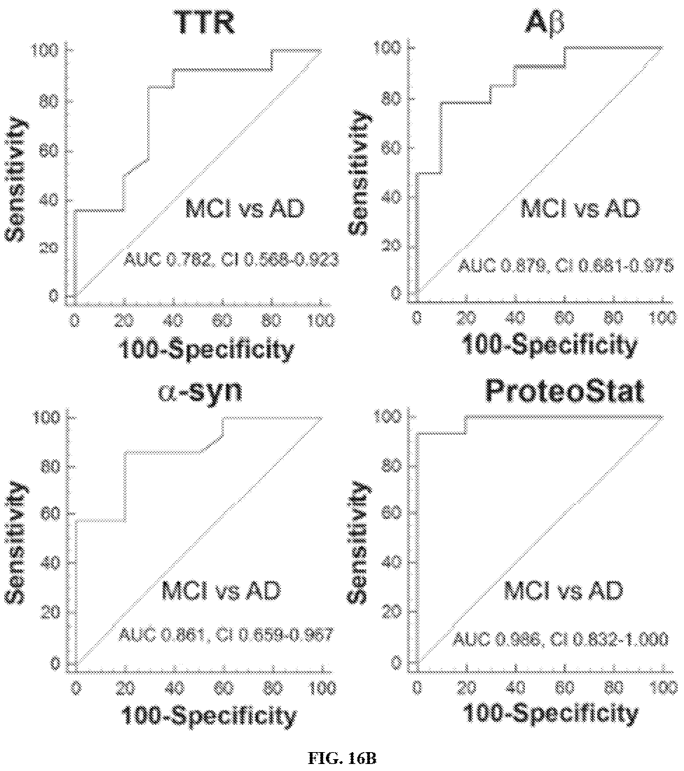

To determine whether Aβ, phosphorylated tau protein, TTR and α-syn are components of protein aggregates, a dual staining with specific antibodies and ProteoStat dye in MCI or AD serum-treated ADTs was performed. Tau harbors multiple potential phosphorylation sites, with the site at tau231 (T231) crucially important for the role of tau in regulation of microtubule binding and involvement in neurodegenerative diseases. Thus, the focus of the screening study was with regard to phosphorylated. A significant amount of Aβ, TTR, α-syn, and phosphorylated T231 immunofluorescence signals were observed, with co-localization of these proteins indicated by a robust ProteoStat signal in MCI or AD serum-treated ADTs, which was absent in the corresponding controls, demonstrating that these proteins are the components of the aggregate complexes (FIGS. 14A-14B and FIG. 15). Quantification analysis demonstrated that AD sera contained higher levels of Aβ and α-syn and a lower level of TTR aggregates than MCI sera (FIG. 16A). FIG. 16B shows the ROC curve analysis with robust differences in TTR, Aβ, and α-syn between MCI and AD serum samples. The AUC and CI values are all significant for TTR, Aβ and α-syn in distinguishing MCI from AD patients.

### Example 6: Detection of Serum-Associated Protein Aggregates in ADT Cells is Not Associated with Transcriptional Induction of Protein Components

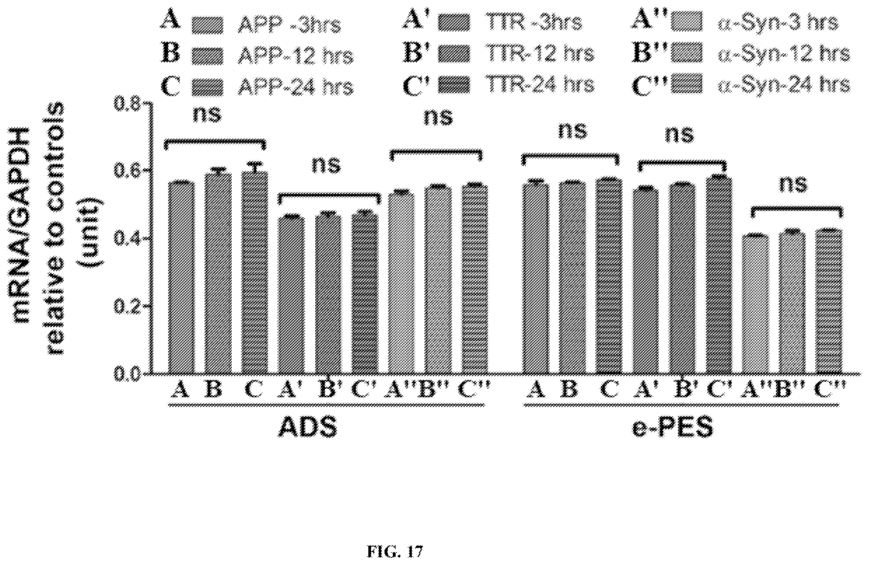

Robust detection of protein aggregates in serum-exposed ADT cells raises the question whether or not mRNA expression for the individual protein components was induced in response to serum from PE or AD patients, which then contributed to excess protein production, leading to protein aggregation. Amyloid precursor protein (APP), TTR, and α-syn transcripts were chosen for analysis using real-time PCR (RT-PCR). RNA was extracted from ADTs treated with serum samples from AD and e-PE patients at 3, 12, and 24 hr. Exposure to PE or AD sera did not significantly alter the mRNA levels of these proteins over the time course (p>0.05) (FIG. 17). Thus, aggregated proteins are internalized from sera and accumulated over time inside ADTs, rather than produced endogenously by the cells.

### Example 7: Treatment of Alzheimer's Disease (AD) with Trehalose in hTAU Transgenic Mice

The efficacy with which trehalose inhibits the onset of Alzheimer's disease (AD) features was assessed using the hTAU transgenic mouse model at ages of 3, 6, 9, and 12 months. The onset of AD features were preempted by the administration of serum obtained from pregnant women with normal pregnancy or preeclampsia. Initial observations suggest serum from subjects with preeclampsia injected at 1 month (30 day old mice) can induce molecular and behavioral features of AD. Additionally, weekly administration of trehalose (2 mg/kg) at 2 months (60 day old mice) for a period of one month inhibited the onset of AD features.

Control studies were performed, wherein a placebo was administered to hTAU-preeclampsia serum-treated mice and the results indicated that AD-associated features were not inhibited.

Furthermore, AD serum was found to cause protein aggregation in human trophoblasts and SHSY neuronal cells, which could be inhibited with administration of trehalose (50 mM) in a 24 h culture experiment. Primary human trophoblasts and SHSY cells were cultured and washed with serum-free media at 70% confluency. The cells were then cultured with 10% serum samples from AD patients for 72 hours (trophoblasts) and 24 hours (neuronal cells), respectively, in the absence or presence of 50 mM trehalose. Cells were stained with ProteoStat dye and evaluated for accumulation of protein aggregates.

### Example 8: Effect of Trehalose on Preeclampsia Serum-Induced Early Onset of AD-Like Features

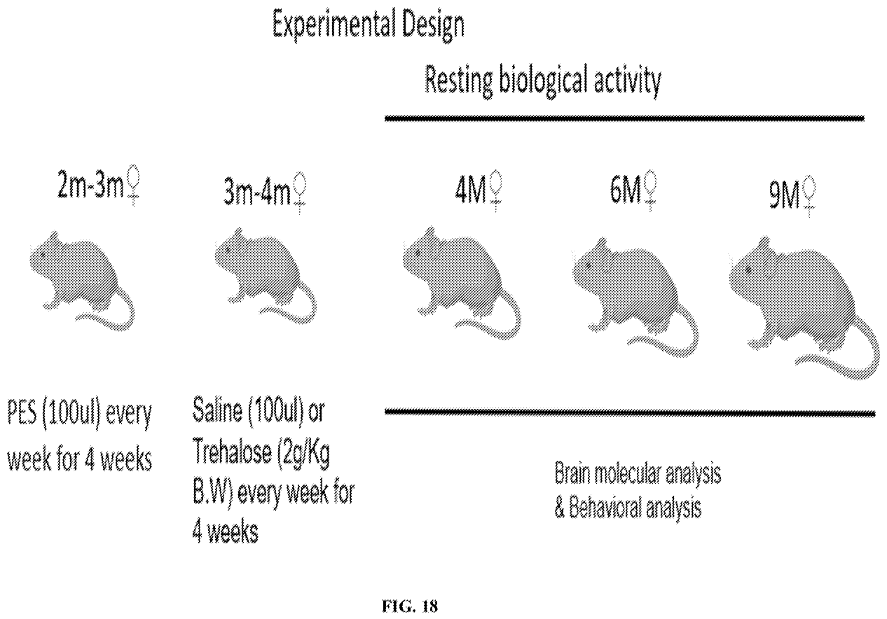

100 μL e-PES (early-onset preeclampsia serum) was injected in 10-12 weeks old non-pregnant female hTAU mice. The i.p. injection was performed once a week for 4 weeks. Trehalose treatment (2 g/kg/week) or saline (100 μL/week) was initiated after completion of 4 weeks of e-PES injection cycle. At the end of the trehalose treatment, a group of mice (n=5 for each group) was sacrificed after 2 weeks of resting biological activity (FIG. 18).

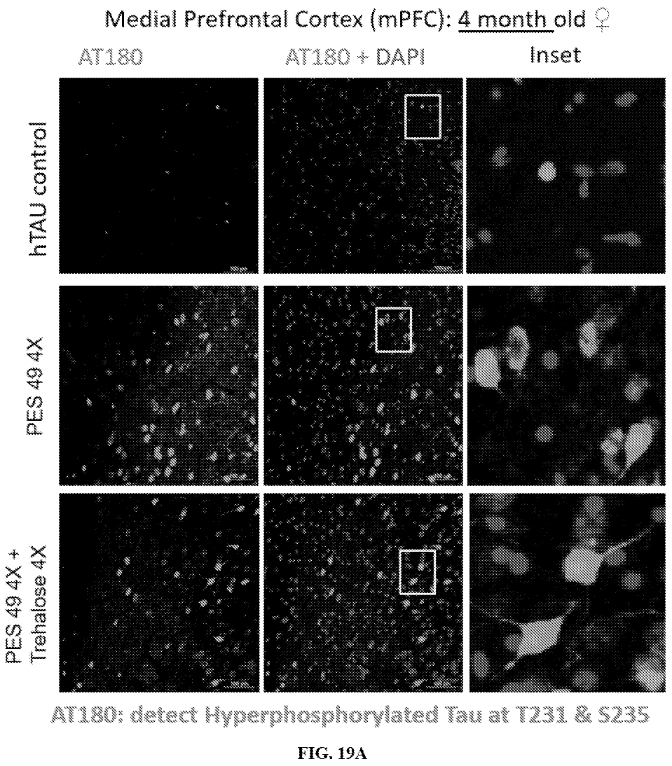

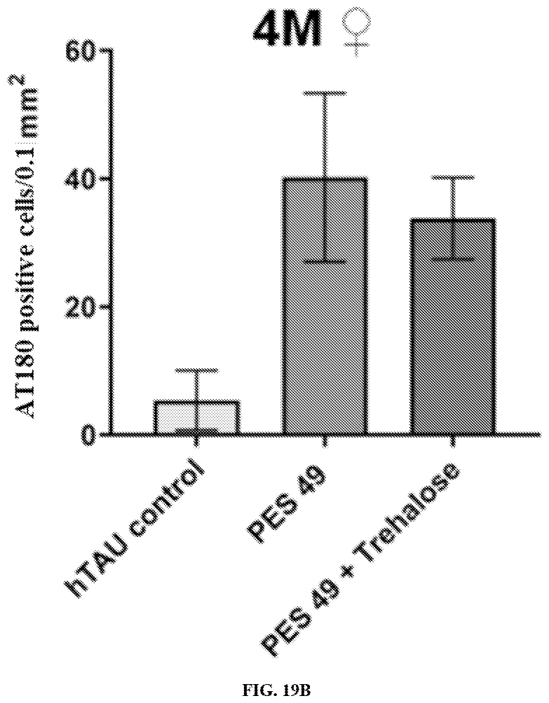

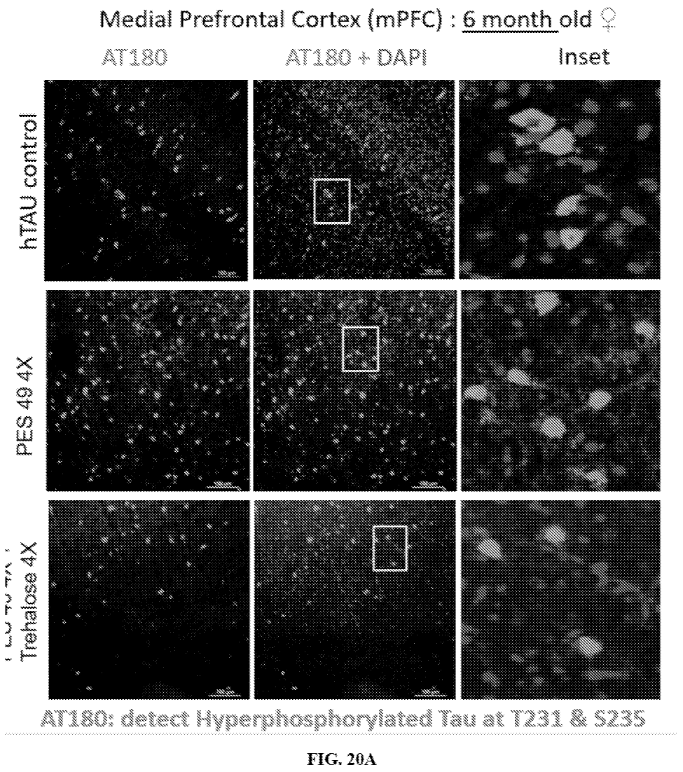

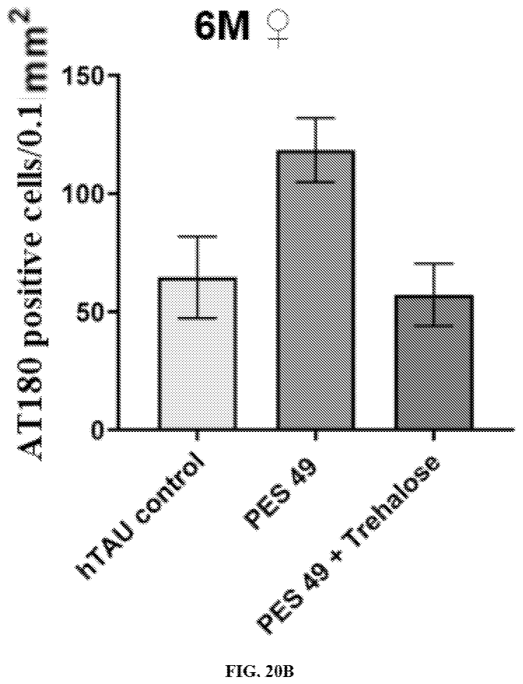

Brain pathologies were evaluated in a group of mice at 4 and 6 months of age. The data demonstrate that preeclamspia serum induced early brain pathological changes at 4 and 6 months of age (FIGS. 19A-19B and FIGS. 20A-20B).

Another two groups were kept to assess their behavioral and brain pathological outcomes (i.e., nesting behavior and/or sensory motor coordination).

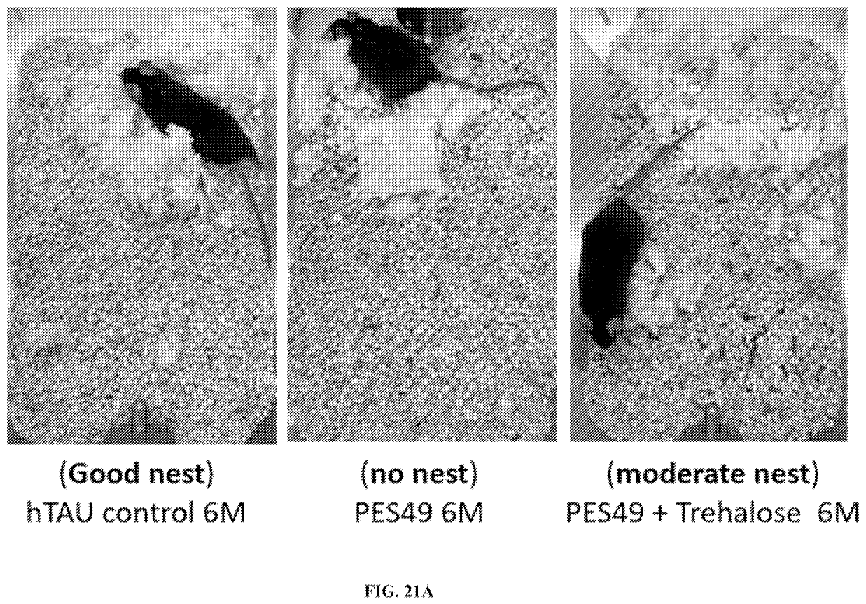

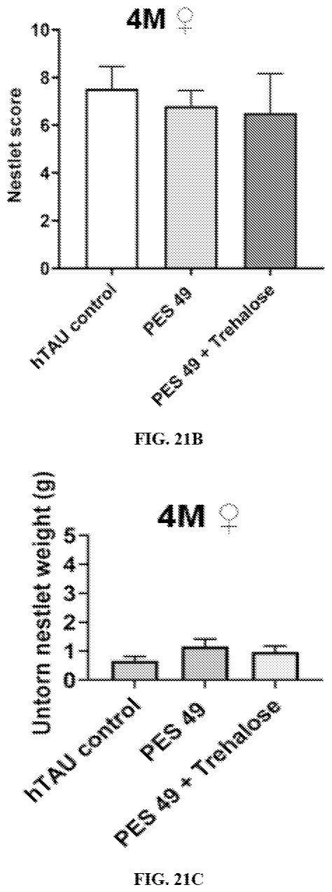

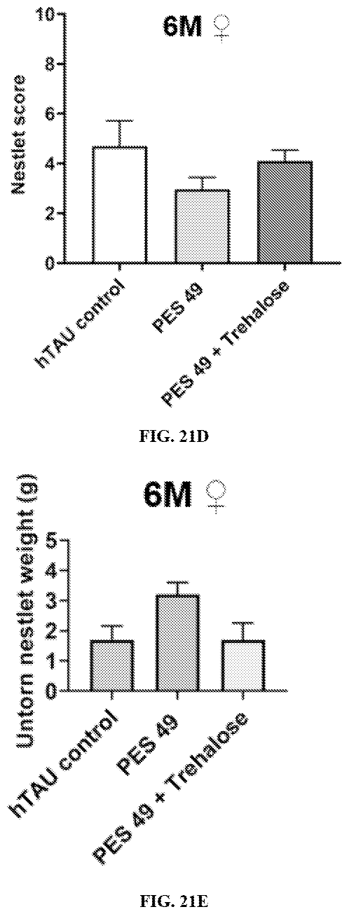

Regarding nesting behavior, there were no significant changes in the nestle score at 4 months of age between control and indicated treatment groups, but e-PES treated hTAU mice showed higher uniform untorn nestlet, indicating poor cognition (FIG. 21A). Further nestlet activity analysis at 6 months showed age-related decline of cognition even in control hTAU mice. Unlike 4 month old hTAU mice, 6 month hTAU mice treated with e-PES alone (n=5) exhibited significantly lower nestlet scores and significantly greater untorn nestlet weights indicative of poor nesting activity compared to control and e-PES+trehalose treated hTAU mice (n=5) (FIGS. 21B-21E). Although limited (4 weeks) treatment with trehalose reduced hTAU burdern and improved cognitive decline.

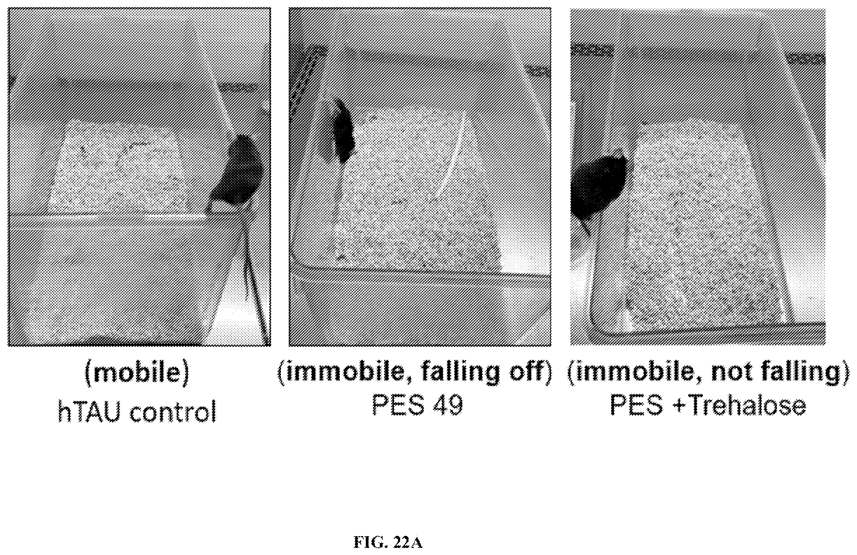

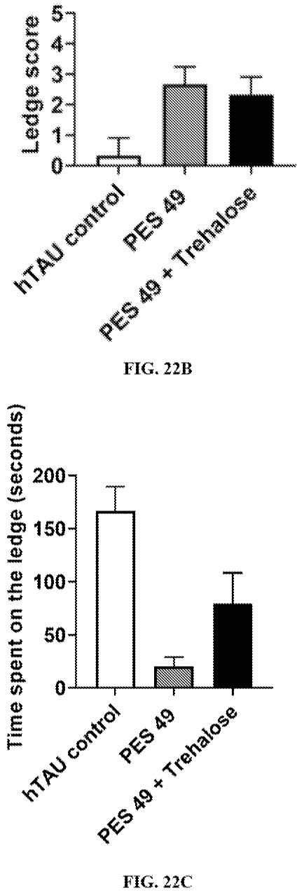

Regarding sensorimotor deficits, ePES injection at 10-12 weeks of age (n=5) accelerated sensorimotor coordination deficits, as detected by a ledge assay. e-PES injection alone increased the ledge score, as the mice were falling off of the ledge repeatedly and decreased the time spent on the ledge (FIG. 22A). Compared to the cognitive test, hTAU mice (n=5) still had significant sensorimotor coordination, as shown by a low ledge score and higher time spend on the ledge (FIG. 22B). Treatment with trehalose partially prevented sensorimotor coordination deficits, as detected by the lede assay (e.g., time spent on the ledge), but could not restore motor activity completely. Trehalose treated mice (n=5) spent significantly more time on the ledge without moving compared to only e-PES treated mice, but they did not fall off of the ledge frequently (FIG. 22C).

Sequence Listing
SEQ ID NO. 1
CAAGCAGTGCAAGACCCATC
SEQ ID NO. 2
AGAAGGGCATCACTTACAAACTC
SEQ ID NO. 3
TGGGAGCCATTTGCCTCTG
SEQ ID NO. 4
AGCCGTGGTGGAATAGGAGTA
SEQ ID NO. 5
TGGTGAGCGAAACAGAAGCC
SEQ ID NO. 6
TCCATAGCAACCTGCGTAATGAA

### Enumerated Embodiments

The following exemplary embodiments are provided, the numbering of which is not to be construed as designating levels of importance:

Embodiment 1 provides a method of detecting a proteinopathy in a subject, the method comprising:
incubating autophagy-deficient trophoblast (ADT) cells in a medium comprising a serum sample collected from the subject to provide ADT cells comprising at least one protein aggregate;
staining the cells comprising at least one protein aggregate with a protein aggregate dye to provide stained cells; and
detecting the at least one protein aggregate in the stained cells.

Embodiment 2 provides the method of Embodiment 1, wherein the proteinopathy is at least one selected from the group consisting of Alzheimer's disease (AD), mild cognitive impairment (MCI), preeclampsia (PE), Lewy body dementia (LBD), gestational diabetes, and Huntington's disease (HD).

Embodiment 3 provides the method of any of Embodiments 1-2, wherein the ADT cells comprise immortalized HchEpC1b human extravillous trophoblast cells.

Embodiment 4 provides the method of Embodiment 3, wherein the immortalized HchEpC1b cells are human papillomavirus E6 (HPV E6) and human telomerase reverse transcriptase (hTERT) transfected.

Embodiment 5 provides the method of any of Embodiments 3-4, wherein the immortalized HchEpC1b cells are stably transfected with a mutant expression vector.

Embodiment 6 provides the method of Embodiment 5, wherein the mutant expression vector is pMRX-IRES-puro-mStrawberry-Atg4BC74A.

Embodiment 7 provides the method of any of Embodiments 5-6, wherein the mutant expression vector inhibits at least one of:
(a) microtubule-associated proteins 1A/1B light chain 3B (MAP1LC3B-II) formation;
(b) autophagy flux; and
(c) lysosomal biogenesis.

Embodiment 8 provides the method of any of Embodiments 1-2, wherein the ADT cells comprise human trophoblast TCL1 or HTR8 cells.

Embodiment 9 provides the method of Embodiment 8, wherein the TCL1 or HTR8 cells are stably transfected with at least one mutant autophagy gene.

Embodiment 10 provides the method of Embodiment 9, wherein the at least one mutant autophagy gene is selected from the group consisting of ATG4B, ATG7, and ATG13.

Embodiment 11 provides the method of any of Embodiments 1-10, wherein the serum sample comprises 10% (v/v) of the ADT cell incubation medium.

Embodiment 12 provides the method of any of Embodiments 1-11, wherein the at least one protein aggregate comprises at least one protein selected from the group consisting of transthyretin (TTR), amyloid β (Aβ), α-synuclein (α-syn), Tau231 (T231), and cis P-Tau.

Embodiment 13 provides the method of any of Embodiments 1-12, wherein the staining comprises incubation of the cells comprising the at least one protein aggregate with the protein aggregate dye for about 10 to 15 minutes.

Embodiment 14 provides the method of any of Embodiments 1-13, wherein the staining comprises the following:
(a) treating the cells comprising the at least one protein aggregate with a phosphate buffered saline (PBS) solution comprising formaldehyde to provide fixed cells;
(b) incubating the fixed cells with glycine to provide quenched cells;
(c) incubating the quenched cells with a permeabilizing solution to provide permeabilized cells;
(d) washing the permeabilized cells to provide washed cells; and
(e) incubating the washed cells with the protein aggregate dye to provide stained cells.

Embodiment 15 provides the method of Embodiment 14, wherein the PBS solution comprising formaldehyde is 4% formaldehyde (wt %).

Embodiment 16 provides the method of any of Embodiments 14-15, wherein the fixed cells are incubated with glycine for about 5 minutes.

Embodiment 17 provides the method of any of Embodiments 14-16, wherein the incubation of the quenched cells with the permeabilizing solution occurs with at least one of:
(a) a period of about 30 minutes; and
(b) a temperature of 0° C.

Embodiment 18 provides the method of any of Embodiments 14-17 wherein the permeabilizing solution comprises at least one of 0.5% Triton X-100 and 3 mM EDTA in PBS.

Embodiment 19 provides the method of any of Embodiments 14-18, wherein the permeabilized cells are washed with a PBS solution.

Embodiment 20 provides the method of any of Embodiments 14-19, wherein the incubation of the washed cells occurs with at least one of:
(a) a period of time of about 20 minutes;
(b) an ambient temperature; and
(c) an absence of light exposure.

Embodiment 21 provides the method of any of Embodiments 1-20, wherein the protein aggregate dye is ProteoStat dye.

Embodiment 22 provides the method of any of Embodiments 1-21, wherein detection of the at least one protein aggregate in the stained cells comprises measuring at least one optical property of the stained cells.

Embodiment 23 provides the method of Embodiment 22, wherein the at least one optical property is fluorescence.

Embodiment 24 provides the method of Embodiment 23, wherein the fluorescence is measured with a confocal microscope.

Embodiment 25 provides the method of Embodiment 24, wherein the confocal microscope is equipped with a 598 red filter set.

Embodiment 26 provides the method of any of Embodiments 24-25, wherein the fluorescence signal is generated using an excitation wavelength of about 550 nm.

Embodiment 27 provides the method of any of Embodiments 24-26, wherein the fluorescence signal is measured with an emission filter of about 600 nm.

Embodiment 28 provides a method of treating, preventing, and/or ameliorating a proteinopathy in a subject in need thereof, the method comprising administering to the subject a pharmaceutical composition comprising a therapeutically effective amount of trehalose or salt, solvate, stereoisomer, derivative, prodrug and any mixtures thereof.

Embodiment 29 provides the method of Embodiment 28, wherein the trehalose stereoisomer is lactotrehalose.

Embodiment 30 provides the method of any of Embodiments 28-29, wherein the proteinopathy is selected from the group consisting of Alzheimer's disease (AD) and mild cognitive impairment (MCI).

Embodiment 31 provides the method of any of Embodiments 28-30, wherein the trehalose administration promotes degradation of at least one protein aggregate.

Embodiment 32 provides the method of Embodiment 31, wherein the at least one degraded protein aggregate comprises at least one protein selected from the group consisting of transthyretin (TTR), amyloid β (Aβ), α-synuclein (α-syn), Tau231 (T231), and cis P-Tau.

Embodiment 33 provides the method of any of Embodiments 28-32, wherein the trehalose is administered to the subject weekly.

Embodiment 34 provides the method of any of Embodiments 28-33, wherein the trehalose is administered to the subject for a period of one month.

Embodiment 35 provides the method of any of Embodiments 28-34, wherein the therapeutically effective amount is about 2 g/kg based on subject weight.

Embodiment 36 provides the method of any of Embodiments 28-35, wherein the pharmaceutical composition comprises trehalose and at least one pharmaceutically acceptable carrier.

Embodiment 37 provides the method of any of Embodiments 28-36, wherein the subject is co-administered at least one additional agent useful for treating, preventing, and/or ameliorating a proteinopathy in a subject.

Embodiment 38 provides the method of Embodiment 37, wherein the at least one additional reagent useful for treating, preventing, and/or ameliorating a proteinopathy is selected from the group consisting of transthyretin (TTR) and human chorionic gonadotropin (hCG).

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of detecting a proteinopathy in a subject, the method comprising:
incubating autophagy-deficient trophoblast (ADT) cells in a medium comprising a serum sample collected from the subject to provide ADT cells comprising at least one protein aggregate;
staining the cells comprising at least one protein aggregate with a protein aggregate dye to provide stained cells; and
detecting the at least one protein aggregate in the stained cells.

2. The method of claim 1, wherein the proteinopathy is at least one selected from the group consisting of Alzheimer's disease (AD), mild cognitive impairment (MCI), preeclampsia (PE), Lewy body dementia (LBD), gestational diabetes, and Huntington's disease (HD).

3. The method of claim 1, wherein the ADT cells comprise immortalized HchEpC1b human extravillous trophoblast cells.

4. The method of claim 3, wherein the immortalized HchEpC1b cells are human papillomavirus E6 (HPV E6) and human telomerase reverse transcriptase (hTERT) transfected.

5. The method of claim 3, wherein the immortalized HchEpC1b cells are stably transfected with a mutant expression vector.

6. The method of claim 5, wherein the mutant expression vector is pMRX-IRES-puro-mStrawberry-Atg4BC74A.

7. The method of claim 5, wherein the mutant expression vector inhibits at least one of:
(a) microtubule-associated proteins 1A/1B light chain 3B (MAP1LC3B-II) formation;
(b) autophagy flux; and
(c) lysosomal biogenesis.

8. The method of claim 1, wherein the ADT cells comprise human trophoblast TCL1 or HTR8 cells.

9. The method of claim 8, wherein the TCL1 or HTR8 cells are stably transfected with at least one mutant autophagy gene.

10. The method of claim 9, wherein the at least one mutant autophagy gene is selected from the group consisting of ATG4B, ATG7, and ATG13.

11. The method of claim 1, wherein the serum sample comprises 10% (v/v) of the ADT cell incubation medium.

12. The method of claim 1, wherein the at least one protein aggregate comprises at least one protein selected from the group consisting of transthyretin (TTR), amyloid β (Aβ), α-synuclein (α-syn), Tau231 (T231), and cis P-Tau.

13. The method of claim 1, wherein the staining comprises incubation of the cells comprising the at least one protein aggregate with the protein aggregate dye for about 10 to 15 minutes.

14. The method of claim 1, wherein the staining step comprises the following:
(a) treating the cells comprising the at least one protein aggregate with a phosphate buffered saline (PBS) solution comprising formaldehyde to provide fixed cells;
(b) incubating the fixed cells with glycine to provide quenched cells;
(c) incubating the quenched cells with a permeabilizing solution to provide permeabilized cells;
(d) washing the permeabilized cells to provide washed cells; and
(e) incubating the washed cells with the protein aggregate dye to provide stained cells.

15. The method of claim 14, wherein the PBS solution comprising formaldehyde is 4% formaldehyde (wt %).

16. The method of claim 14 or 15, wherein the fixed cells are incubated with glycine for about 5 minutes.

17. The method of claim 14, wherein the incubation of the quenched cells with the permeabilizing solution occurs with at least one of:
(a) a period of about 30 minutes; and
(b) a temperature of 0° C.

18. The method of claim 14, wherein the permeabilizing solution comprises at least one of 0.5% Triton X-100 and 3 mM EDTA in PBS.

19. The method of claim 14, wherein the permeabilized cells are washed with a PBS solution.

20. The method of claim 14, wherein the incubation of the washed cells occurs with at least one of:
(a) a period of time of about 20 minutes;
(b) an ambient temperature; and
(c) an absence of light exposure.

21. The method of claim 1, wherein the protein aggregate dye is ProteoStat dye.

22. The method of claim 1, wherein detection of the at least one protein aggregate in the stained cells comprises measuring at least one optical property of the stained cells.

23. The method of claim 22, wherein the at least one optical property is fluorescence.

24. The method of claim 23, wherein the fluorescence is measured with a confocal microscope.

25. The method of claim 24, wherein the confocal microscope is equipped with a 598 red filter set.

26. The method of claim 23, wherein the fluorescence signal is generated using an excitation wavelength of about 550 nm.

27. The method of claim 23, wherein the fluorescence signal is measured with an emission filter of about 600 nm.

28. A method of treating, preventing, and/or ameliorating a proteinopathy in a subject in need thereof, the method comprising administering to the subject a pharmaceutical composition comprising a therapeutically effective amount of trehalose or salt, solvate, stereoisomer, derivative, prodrug and any mixtures thereof.

29. The method of claim 28, wherein the trehalose stereoisomer is lactotrehalose.

30. The method of claim 28, wherein the proteinopathy is selected from the group consisting of Alzheimer's disease (AD) and mild cognitive impairment (MCI).

31. The method of claim 28, wherein the trehalose administration promotes degradation of at least one protein aggregate.

32. The method of claim 31, wherein the at least one degraded protein aggregate comprises at least one protein selected from the group consisting of transthyretin (TTR), amyloid β (Aβ), α-synuclein (α-syn), Tau231 (T231), and cis P-Tau.

33. The method of claim 28, wherein the trehalose is administered to the subject weekly.

34. The method of claim 28, wherein the trehalose is administered to the subject for a period of one month.

35. The method of claim 28, wherein the therapeutically effective amount is about 2 g/kg based on subject weight.

36. The method of claim 28, wherein the pharmaceutical composition comprises trehalose and at least one pharmaceutically acceptable carrier.

37. The method of claim 28, wherein the subject is co-administered at least one additional agent useful for treating, preventing, and/or ameliorating a proteinopathy in a subject.

38. The method of claim 37, wherein the at least one additional reagent useful for treating, preventing, and/or ameliorating a proteinopathy is selected from the group consisting of transthyretin (TTR) and human chorionic gonadotropin (hCG).


SEQUENCE LISTING (6 sequences)

SEQ ID NO: 1 - DNA, 20 bp, Artificial Sequence
  Chemically synthesized
caagcagtgcaagacccatc

SEQ ID NO: 2 - DNA, 23 bp, Artificial Sequence
  Chemically synthesized
agaagggcatcacttacaaactc

SEQ ID NO: 3 - DNA, 19 bp, Artificial Sequence
  Chemically synthesized
tgggagccatttgcctctg

SEQ ID NO: 4 - DNA, 21 bp, Artificial Sequence
  Chemically synthesized
agccgtggtggaataggagta

SEQ ID NO: 5 - DNA, 20 bp, Artificial Sequence
  Chemically synthesized
tggtgagcgaaacagaagcc

SEQ ID NO: 6 - DNA, 23 bp, Artificial Sequence
  Chemically synthesized
tccatagcaacctgcgtaatgaa